(19) United States
(12) Patent Application Publication
(10) Pub. No.: US 2023/0099519 A1
(43) Pub. Date: Mar. 30, 2023

(54) SYSTEMS AND METHODS FOR MANAGING STRESS EXPERIENCED BY USERS DURING EVENTS

(71) Applicant: Fierce, Inc., Seattle, WA (US)

(72) Inventor: Edward J. Beltran, Seattle, WA (US)

(73) Assignee: Fierce, Inc., Seattle, WA (US)

(21) Appl. No.: 17/696,429

(22) Filed: Mar. 16, 2022

Related U.S. Application Data
(60) Provisional application No. 63/248,348, filed on Sep. 24, 2021.

(51) Int. Cl. A61B 5/16; G16H 50/30; G16H 20/00; G16H 10/20; H04L 51/02

(52) U.S. Cl. CPC A61B 5/165; G16H 50/30; G16H 20/00; G16H 10/20; H04L 51/02

(57) ABSTRACT

The present invention is directed to a platform for the tracking and management of stress encountered by a user. Biometric data corresponding to the stress of the user is collected and analyzed to determine the stress level of a user. In one embodiment, this data is cross-referenced with data such as data from a user's calendar operable to provide context and/or reason to the stress level. Based on the data, conversation models and resources are operable to be provided to the user to help in mitigating and managing stress.

### CROSS REFERENCES TO RELATED APPLICATIONS

This application is related to and claims priority from the following U.S. patent applications: this application claims priority to and the benefit of U.S. Provisional Patent Application No. 63/248,348 filed Sep. 24, 2021, which is incorporated herein by reference in its entirety.

### BACKGROUND OF THE INVENTION

### 1. Field of the Invention

The present invention relates generally to the field of data processing. More specifically, the present invention relates to systems and methods for facilitating stress management during events.

### 2. Description of the Prior Art

It is generally known in the prior art to provide stress management techniques.

U.S. Pat. No. 11,191,433 for Apparatus and method for a personalized reminder with intelligent self-monitoring by inventors Brancaccio, et al., filed Feb. 17, 2018, and issued Dec. 7, 2021, is directed to a system and method disc that collects user reported, self-monitored On-task/Off-task Behavior, Fidgeting Behaviors and Walking/Running behaviors as quantified by motion sensors and an intelligent scheduling system. The collected data tells the reminder device what environment a user is scheduled to be in at any point in time in order to appropriately collect behavioral information and use said information to encourage users to be mindful of their own actions and behaviors in order to increase time spent on-task.

U.S. Pat. No. 9,402,581 for Apparatus and method for improving psychophysiological function for performance under stress by inventors Kusik, et al., filed Aug. 11, 2015, and issued Aug. 2, 2016, is directed to a computer-implemented method for improving psychophysiological function for the performance of a subject under stress including, after a plurality of sensors that monitor stress-indicating physiological parameters have been coupled to the subject, exposing the subject, using computer processes, to at least one training segment during which is determined a degree to which the subject has achieved a targeted level of least one stress-indicating physiological parameter as to be indicative of coherence in the subject. Additionally, the method includes providing, to the subject, feedback indicative of the degree to which the subject has achieved the targeted level of at least one parameter as to be indicative of coherence in the subject.

US Patent Pub. No. 2021/0201696 for Automated speech coaching systems and methods by inventors Perez, et al., filed Jul. 18, 2017, and published Jul. 1, 2021, is directed to a system including data gathering circuitry to collect audio, video, and biometric data generated by a speaker during a presentation. All or a portion of the collected audio, video, and biometric data may be stored or otherwise retained on one or more storage devices. All or a portion of the collected audio, video, and biometric data may be forwarded to the presentation analysis circuitry. The presentation analysis circuitry detects at least one of an audio presentation event; a video presentation event; or a biometric presentation event based at least in part on the collected audio, video, and biometric data received from the data gathering circuitry. The presentation analysis circuitry forwards the detected audio presentation event; a video presentation event; or a biometric presentation event to the presenter feedback circuitry. The presenter feedback circuitry generates feedback for the presentation to the speaker.

U.S. Pat. No. 8,715,179 for Call center quality management tool by inventors Price, et al., filed Feb. 18, 2010, and issued May 6, 2014, is directed to systems and methods for call center quality management. A sensor may monitor a response of a dialogue participant. A reference index may correlate the response to a known condition. A processor device may detect the known condition or a threshold number of known conditions. The processor may relate the known condition to feedback. A feedback mechanism may provide feedback to the participant. The feedback may be provided based on the known condition. The feedback may be configured to improve call center quality. The feedback may be transmitted during a call. The feedback may be based on the threshold number.

U.S. Pat. No. 10,478,131 for Determining baseline contexts and stress coping capacity by inventors Jain, et al., filed Jun. 15, 2016, and issued Nov. 19, 2019, is directed to a method for monitoring a health characteristic of a user based on one or more biological measurements including selecting a context from a plurality of contexts, each context corresponding to a baseline health value, and each context being defined by a plurality of recorded events each comprising one or more of repeated biological states, repeated user activity, or space-time coordinates of the user, and then monitoring the health characteristic of the user based on one or more bio-sensing measurements in comparison to the baseline health value corresponding to the selected context.

U.S. Pat. No. 11,191,466 for Determining mental health and cognitive state through physiological and other non-invasively obtained data by inventors Heneghan, et al., filed Jun. 28, 2019, and issued Dec. 7, 2021, is directed to the use of physiological variables, metrics, biomarkers, and other data points, in connection with a non-invasive wearable device, to screen for, and predict, mental health issues and cognitive states. In addition to metrics such as heart rate, sleep data, activity level, gamification data, and the like, information such as text message and email data, as well as vocal data obtained through a phone and/or a microphone, maybe analyzed, provided user authorization. Applying predictive modeling, one or more of the monitored metrics can be correlated with mental states and disorders. Identified patterns can be used to update the predictive models, such as via machine learning-trained models, as well as to update individual event predictions. Information about the mental state predictions, and updates thereto, can be surfaced to the user accordingly.

US Patent Pub. No. 2019/0115107 for Electronic device and method for providing stress index corresponding to activity of user by inventors Hong, et al., filed Sep. 28, 2018 and published Apr. 18, 2019, is directed to an electronic device is provided that includes a display, a biometric sensor, a motion sensor, a communication circuit configured to receive a signal for obtaining information related to a location of the electronic device, and a processor electrically connected with the display, the biometric sensor, the motion sensor, and the communication circuit, wherein the processor is configured to identify repeated activities related to the user, which follow a lapse of time, based on motion information obtained according to the lapse of time by using the motion sensor and location information obtained according to the lapse of time by using the communication module, calculate a stress index of the user corresponding to the repeated activities based on biometric information obtained by using the biometric sensor, and provide at least one activity of the repeated activities and a stress index corresponding to the at least one activity.

U.S. Pat. No. 10,966,648 for Identifying stress levels associated with context switches by inventors Delaney, et al., filed Feb. 25, 2019, and issued Apr. 6, 2021, is directed to a computer-implemented method that includes: receiving, by a computing device, information identifying a user's activity; determining, by the computing device, the user's tasks based on the information identifying the user's activity; determining, by the computing device, the user's context switches based on the user's tasks; receiving, by the computing device, biometrics data associated with the user via an application programming interface (API); determining, by the computing device, the user's stress levels at various times based on the biometrics data; storing, by the computing device, information linking the user's stress level with the user's context switches; and outputting, by the computing device, the information linking the user's stress level with the user's context switches.

US Patent Pub. No. 2020/0135050 for Internet of things public speaking coach by inventor Nunez, filed Oct. 31, 2018, and published Apr. 30, 2020, is directed to approaches enabling the delivery of real-time internet of things (IoT) feedback to optimize a public speaking performance. More specifically, a set of data representing a speaking performance of a user is captured and analyzed to generate a speaking performance profile of the user. This profile is compared to a reference speaking performance profile and, based on the comparison, a set of performance improvement strategies for the user is generated. A performance improvement strategy is selected from the set of performance improvement strategies based on an identification of availability of a set of IoT devices for delivery of at least one of the strategies. Instructions are then communicated, responsive to the captured speaking performance associated with the user, to an available IoT device to deliver the selected performance improvement strategy to the user through an output user interface of the available IoT device during the speaking performance.

U.S. Pat. No. 11,045,102 for Low noise sensing circuit with cascaded reference by inventors Felix, et al., filed Jun. 17, 2019, and issued Jun. 29, 2021, is directed to cascaded reference circuits and low amplitude signal sensing circuits that are useful in applications where high sensitivity, low noise measurements are needed. Such embodiments may be especially useful in health and fitness wearable devices. Systems incorporate the circuits, as well as methods for using these circuits to determine quantities and qualities of a person's moods, such as how much and what kinds of stress they experience. The provided devices are useful on limbs and appendages, such as in a smartwatch that is worn on the wrist. Methods are provided for using the devices of this disclosure to privately alert wearers to an increase in bad stress at the moment when they can take actions to reduce their stress and physiological stress responses. These devices are useful for measuring and increasing the effectiveness of relaxation techniques. As a result of using such methods and devices, people are healthier, they make more response-able decisions, and relationships improve.

U.S. Pat. No. 9,668,693 for Method for improving psychophysiological function for performance under stress by inventors Kusik, et al., filed Jul. 6, 2016, and issued Jun. 6, 2017, is directed to a method of improving the psychophysiological function of a subject performing a stress-inducing activity using a computer including, after a plurality of sensors that monitor stress-indicating physiological parameters have been coupled to the subject and to the computer, providing, by the computer to the subject, a set of training segments that each present the subject with one or more visual, audible, or tactile prompts, wherein in at least one of the training segments, the prompts induce the subject to simultaneously perform both the stress-inducing activity and a relaxation-inducing protocol. The computer provides the set of training segments until a value of at least one physiological parameter that indicates stress in the subject is within a pre-defined range of a baseline value of the parameter, thereby indicating that the subject has successfully performed the stress-inducing activity while maintaining alertness with a relative minimum of stress.

US Patent Pub. No. 2021/0275072 for Method for predicting arousal level and arousal level prediction apparatus by inventors Kusukame, et al., filed May 24, 2021, and published Sep. 9, 2021, is directed to a method for predicting an arousal level used by a computer of an arousal level prediction apparatus that predicts an arousal level of a user. The method includes obtaining current biological information regarding the user detected by a sensor, and calculating the current arousal level of the user based on the current biological information. The method further includes obtaining current environment information indicating a current environment around the user, and predicting a future arousal level, which is an arousal level a certain period of time later, based on the current arousal level and the current environment information. Based on the predicted future arousal level, the method further issues a notification to the user, or controls an operation of a device.

U.S. Pat. No. 9,691,296 for Methods and apparatus for conversation coach by inventors Hoque, et al., filed Jun. 3, 2014, and issued Jun. 27, 2017, is directed to a display screen and speakers presenting an audiovisual display of an animated character to a human user during a conversational period of a coaching session. The virtual character asks questions, listens to the user, and engages in mirroring and backchanneling. A camera and microphone gather audiovisual data regarding the behavior of the user. After the conversational period, the display screen and speakers display feedback to the user regarding the user's behavior. For example, the feedback may include a plot of the user's smiles over time or information regarding the prosody of the user's speech. The feedback may also include playing a video of the user that was recorded during the conversational period. The feedback may also include a timeline of the human user's behavior. Virtual coaching may be provided over the Internet.

U.S. Pat. No. 9,339,188 for Methods from monitoring health, wellness, and fitness with feedback by inventor Proud, filed Aug. 6, 2013, and issued May 17, 2016, is directed to a method for obtaining monitored information about an individual to create life activity data. Individual information is detected or measured. The individual information is selected from at least one of, an individual's activities, behaviors and habit information, and an individual's health. A monitoring device is used to detect or measure individual information. The monitoring device includes ID circuitry with ID storage that contains a unique individual ID, a communication system that reads and transmits the unique individual ID from the ID storage, a power source, and a pathway system. The individual information is received from the monitoring device at a telemetry system that includes a database. The individual information is analyzed using life activity data with one or more analysis tools at the telemetry system. Life activity data is created for the individual.

US Patent Pub. No. 2019/0392730 for Public speaking trainer with 3-d simulation and real-time feedback by inventors Gupta, et al., filed Sep. 6, 2019, and published Dec. 26, 2019, is directed to a public speaking trainer having a computer system including a display monitor. A microphone is coupled to the computer system. A video capture device is coupled to the computer system. A biometric device is coupled to the computer system. A simulated environment including a simulated audience member is rendered on the display monitor using the computer system. A presentation is recorded onto the computer system using the microphone and video capture device. The first feature of the presentation is extracted based on data from the microphone and video capture device while recording the presentation. A metric is calculated based on the first feature. The simulated audience member is animated in response to a change in the metric. A score is generated based on the metric. The score is displayed on the display monitor of the computer system after recording the presentation. A training video is suggested based on the score.

U.S. Pat. No. 11,006,874 for Real-time stress determination of an individual by inventors Jayaraman, et al., filed Aug. 13, 2013, and issued May 18, 2021, is directed to the a computer-implemented method for real-time determination of stress levels of an individual. The method includes receiving at least one stream of physiological data from at least one primary sensor for a predetermined duration and preprocessing at least one stream of physiological data to extract physiological parameters, whereas the preprocessing includes performing a preliminary analysis on at least one stream of physiological data. The method further includes determining a stress level of the individual based on at least the physiological parameters, wherein the determining comprises performing a statistical analysis on the physiological parameters.

US Patent Pub. No. 2021/0000355 for Stress evaluation device, stress evaluation method, and non-transitory computer-readable medium by inventors Zukawa, et al., filed Sep. 23, 2020, and published Jan. 7, 2021, is directed to a stress evaluation device including a first sensor that measures a heart rate and a heart rate variability of a measurement subject; a calculator that calculates (i) an amount of change in heart rate and (ii) an amount of change in heart rate variability; and a determiner that determines a factor for the stress of the measurement subject in accordance with the (i) and the (ii) and that outputs a determination result. The amount of change in heart rate is an amount of change from a reference value to the measured heart rate. The amount of change in heart rate variability is an amount of change from a reference value to the measured heart rate variability. The determiner makes (I) a comparison between the (i) and a first threshold, and (II) a comparison between the (ii) and a second threshold to determine the factor for the stress.

U.S. Pat. No. 10,692,606 for Stress level reduction using haptic feedback by inventors Bender, et al., filed Oct. 23, 2018, and issued Jun. 23, 2020, is directed to methods, computer program products, and systems. The method computer program products and systems can include, for instance: obtaining biometric data of a first user, the first user using a first client computing device associated to the first user; returning a current stress level classification of the first user in dependence on the processing of the biometric data; generating feedback data in dependence on the current stress level classification of the first user, the feedback data including haptic response feedback data; and sending the feedback data to a second client computing device of a second user to present feedback to the second user, the feedback is in dependence on the current stress level classification of the first user and including haptic feedback.

U.S. Pat. No. 10,734,103 for Stress management system and stress management method by inventors Kaneko, et al., filed Aug. 21, 2017, and issued Aug. 4, 2020, is directed a stress management system that manages psychological stress of a user. The system includes a first sensor that detects biological data of a user; a second sensor that detects lifelog data indicating an activity history of the user; a generator that generates stress data using the biological data, the stress data indicating a time-series variation in a stress level of the user; an estimator that, when the stress level included in the stress data exceeds a threshold value, estimates whether or not stress experienced by the user is interpersonal stress that is caused by contact with other people, using the life log data; and a notifier that notifies the user of a result of the estimation by the estimator.

U.S. Pat. No. 9,072,478 for System and method for improving presentation skills by inventor Feerst, filed Jun. 10, 2014, and issued Jul. 7, 2015, is directed to a system and method for improving social and presentation skills of persons with a social communication disorder such as Autism. Social anxiety or a lack of confidence, the method including rendering and displaying on a display device a presentation script for an oral presentation to be made by a user; monitoring eye movement of the user during the oral presentation to measure a user's pupil movement and/or a user's gaze direction; displaying an indicia on a separate display screen and/or an upper portion of the display device; periodically displaying within the presentation script a visual prompt to cue the user to look at the indicia; measuring eye movement using eye-tracking software and/or an eye tracking device, at the occurrence of the visual prompt; and evaluating whether the user made eye contact with the indicia when prompted.

U.S. Pat. No. 11,120,405 for System and method for interview training with time-matched feedback by inventors Steinhoff, et al., filed Sep. 17, 2020, and issued Sep. 14, 2021, is directed to interviewing training and providing interview feedback. An exemplary method comprises: at an electronic device that is in communication with a display and one or more input devices: receiving, via the one or more input devices, media data corresponding to a user's responses to a plurality of prompts; analyzing the media data; and while displaying, on the display, a media representation of the media data, displaying a plurality of analysis representations overlaid on the media representation, wherein each of the plurality of analysis representations is associated with an analysis of content located at a given time in the media representation and is displayed in coordination with the given time in the media representation.

US Patent Pub. No. 2014/0221787 for System for monitoring and presenting health, wellness and fitness data having user-selectable parameter by inventors Teller, et al., filed Apr. 9, 2014, and published Aug. 7, 2014, is directed to a wireless communications device, such as the cellular telephone, having sensors to generate data indicative of physiological or contextual parameters of a user. A processor on the wireless communications device is adapted to derive physiological state information of the user from the contextual or physiological parameters. The apparatus may include a central monitoring unit remote from the sensors for storing data and transmitting data to a recipient.

US Patent Pub. No. 2014/0330094 for System for monitoring and presenting health, wellness and fitness utilizing multiple user trend data having user-selectable parameters by inventors Pacione, et al., filed Jul. 21, 2014, and published Nov. 6, 2014, is directed to a nutrition and activity management system that monitors energy expenditure of an individual through the use of a body-mounted sensing apparatus. The apparatus is particularly adapted for continuous wear. The system is also adaptable or applicable to measuring a number of other physiological parameters and reporting the same and derivations of such parameters. A weight management embodiment is directed to achieving an optimum or preselected energy balance between calories consumed and energy expended by the user. An adaptable computerized nutritional tracking system is utilized to obtain data regarding food consumed, Relevant and predictive feedback is provided to the user regarding the mutual effect of the user's energy expenditure, food consumption and other measured or derived or manually input physiological contextual parameters upon progress toward a said goal.

U.S. Pat. No. 10,835,168 for Systems and methods for estimating and predicting emotional states and affects and providing real-time feedback by inventor Flickinger, filed Jun. 24, 2017, and issued Nov. 17, 2020, is directed to systems and methods for estimating emotional states, moods, affects of an individual and providing feedback to the individual or others. Systems and methods that provide real-time detection and monitoring of physical aspects of an individual and/or aspects of the individual's activity and means of estimating that person's emotional state or affect and change to those are also disclosed. Real-time feedback to the individual about the person's emotional change, change or potential change is provided to the user, helping the user cope, adjust or appropriately act on their emotions.

U.S. Pat. No. 9,138,186 for Systems for inducing change in a performance characteristic by inventors Price, et al., filed Feb. 18, 2010, and issued Sep. 22, 2015, is directed to systems and methods for automated on-boarding of a new-hire. A sensor may monitor the response of the new hire. A reference index may correlate the response to a known condition. A processor device may detect the known condition or a threshold number of known conditions. The processor may relate the known condition to feedback. A feedback mechanism may provide feedback to the new hire. The feedback may be provided based on the known condition. The feedback may be configured to provide the new hire with directions, a policy reminder, or any other suitable information. The feedback may be based on the threshold number.

### SUMMARY OF THE INVENTION

The present invention relates to systems and methods for stress reduction in users and more specifically to platforms for analyzing and implementing data from multiple sources to identify and treat stress in individuals.

It is an object of this invention to provide a platform to manage and reduce stress during high-stress situations by recommending conversation models and other resources to help navigate the situation.

In one embodiment, the present invention includes a system for stress management including an application on at least one electronic device including a memory and a processor configured for network communication with at least one server computer comprising at least one database, wherein the application is further operable to receive biometric data collected by at least one body sensor, wherein the application is operable to analyze the biometric data and determine a stress level associated with the biometric data, wherein the application is operable to receive calendar data from at least one calendar data source, wherein the at least one server computer is operable to receive the biometric data and the calendar data from the application, wherein the at least one server computer is operable to determine and retrieve at least one recommended resource from the at least one database based on the stress level and/or the calendar data, wherein the at least one server computer is operable to send the at least one recommended resource to the application, and wherein the application is operable to present the at least one recommended resource via a graphical user interface (GUI).

In another embodiment, the present invention includes a system for stress management including an application on at least one electronic device including a memory and a processor configured for network communication with at least one server computer comprising at least one database, wherein the application is further operable to receive biometric data collected by at least one body sensor, wherein the application is operable to analyze the biometric data and determine a stress level associated with the biometric data, wherein the application is operable to receive chat data from the at least one electronic device, wherein the at least one server computer is operable to receive the biometric data and the chat data from the application, wherein the at least one server computer is operable to determine and retrieve at least one recommended resource from the at least one database using an artificial intelligence engine and/or a machine learning engine based on the stress level and/or the chat data, wherein the at least one server computer is operable to send the at least one recommended resource to the application, and wherein the at least one server computer is operable to send the at least one recommended resource to the application

In yet another embodiment, the present invention includes a method for stress management including an application on an electronic device including a processor and a memory receiving data from at least one data source, wherein the data includes biometric data and calendar data, the application analyzing the biometric data to determine a stress level associated with the data, a server computer in network communication with the application on the electronic device determining and retrieving at least one recommended resource based on the data, the server computer sending the at least one recommended resource to the application, and the application presenting the at least one recommended resource via a GUI, and wherein the biometric data is collected by at least one body sensor.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1 illustrates a block diagram of a system for managing stress experienced during events according to one embodiment of the present invention.

FIG. 2 illustrates a method for connecting a body sensor data source to the platform according to one embodiment of the present invention.

FIG. 3 illustrates a method for connecting a calendar application data source to the platform according to one embodiment of the present invention.

FIG. 4 illustrates a method for managing stress based on conversations according to one embodiment of the present invention.

FIG. 5 illustrates a method for recommending counselors based on user feedback according to one embodiment of the present invention.

FIG. 6 illustrates a method for measuring stress levels according to one embodiment of the present invention.

FIG. 7 illustrates a sign up and login method according to one embodiment of the present invention.

FIG. 8 illustrates a method for accessing content using a chatbot according to one embodiment of the present invention.

FIG. 9 illustrates a method for accessing a stress tracker according to one embodiment of the present invention.

FIG. 10 illustrates an example of an introductory GUI for a stress management platform according to one embodiment of the present invention.

FIG. 11 illustrates an example of a chatbot GUI for a stress management platform according to one embodiment of the present invention.

FIG. 12 illustrates an example of a dashboard GUI for a stress management platform according to one embodiment of the present invention.

FIG. 13 illustrates an example of a stress event detail GUI for a stress management platform according to one embodiment of the present invention.

FIG. 14 illustrates an example of a confrontation conversation model GUI for a stress management platform according to one embodiment of the present invention.

FIG. 15 illustrates an example of a confrontation conversation model GUI for a stress management platform according to one embodiment of the present invention.

FIG. 16 illustrates an example of a confrontation conversation model GUI for a stress management platform according to one embodiment of the present invention.

FIG. 17 illustrates an example of a confrontation conversation model GUI for a stress management platform according to one embodiment of the present invention.

FIG. 18 illustrates an example of a confrontation conversation model GUI for a stress management platform according to one embodiment of the present invention.

FIG. 19 illustrates an example of a confrontation conversation model GUI for a stress management platform according to one embodiment of the present invention.

FIG. 20 illustrates an example of a confrontation conversation model GUI for a stress management platform according to one embodiment of the present invention.

FIG. 21 illustrates an example of a confrontation conversation model GUI for a stress management platform according to one embodiment of the present invention.

FIG. 22 illustrates an example of a confrontation conversation model GUI for a stress management platform according to one embodiment of the present invention.

FIG. 23 illustrates an example of a confrontation conversation model GUI for a stress management platform according to one embodiment of the present invention.

FIG. 24 illustrates an example of a confrontation conversation model GUI for a stress management platform according to one embodiment of the present invention.

FIG. 25 illustrates an example of a confrontation conversation model GUI for a stress management platform according to one embodiment of the present invention.

FIG. 26 illustrates an example of a confrontation conversation model GUI for a stress management platform according to one embodiment of the present invention.

FIG. 27 illustrates an example of a confrontation conversation model GUI for a stress management platform according to one embodiment of the present invention.

FIG. 28 illustrates an example of a confrontation conversation model GUI for a stress management platform according to one embodiment of the present invention.

FIG. 29 illustrates an example of a confrontation conversation model GUI for a stress management platform according to one embodiment of the present invention.

FIG. 30 illustrates an example of a confrontation conversation model GUI for a stress management platform according to one embodiment of the present invention.

FIG. 31 illustrates an example of a confrontation conversation model GUI for a stress management platform according to one embodiment of the present invention.

FIG. 32 illustrates a schematic diagram of a system of the present invention.

### DETAILED DESCRIPTION

The present invention is generally directed to systems and methods for managing stress during events.

In one embodiment, the present invention includes a system for stress management comprising an application on at least one electronic device including a memory and a processor configured for network communication with at least one server computer comprising at least one database, wherein the application is further operable to receive biometric data collected by at least one body sensor, wherein the application is operable to analyze the biometric data and determine a stress level associated with the biometric data, wherein the application is operable to receive calendar data from at least one calendar data source, wherein the at least one server computer is operable to receive the biometric data and the calendar data from the application, wherein the at least one server computer is operable to determine and retrieve at least one recommended resource from the at least one database based on the stress level and/or the calendar data, wherein the at least one server computer is operable to send the at least one recommended resource to the application, and wherein the application is operable to present the at least one recommended resource via a graphical user interface (GUI).

In another embodiment, the present invention includes a system for stress management comprising an application on at least one electronic device including a memory and a processor configured for network communication with at least one server computer comprising at least one database, wherein the application is further operable to receive biometric data collected by at least one body sensor, wherein the application is operable to analyze the biometric data and determine a stress level associated with the biometric data, wherein the application is operable to chat data from the at least one electronic device, wherein the at least one server computer is operable to receive the biometric data and the chat data from the application, wherein the at least one server computer is operable to determine and retrieve at least one recommended resource from the at least one database using an artificial intelligence engine and/or a machine learning engine based on the stress level and/or the chat data, wherein the at least one server computer is operable to send the at least one recommended resource to the application, and wherein the at least one server computer is operable to send the at least one recommended resource to the application.

In yet another embodiment, the present invention includes a method for stress management comprising an application on an electronic device including a processor and a memory receiving data from at least one data source, wherein the data includes biometric data and calendar data, the application analyzing the biometric data to determine a stress level associated with the data, a server computer in network communication with the application on the electronic device determining and retrieving at least one recommended resource based on the data, the server computer sending the at least one recommended resource to the application, and the application presenting the at least one recommended resource via a GUI, and wherein the biometric data is collected by at least one body sensor.

As people interact more often through technology, individuals are reporting higher stress levels during face-to-face conversations. Much of this stress is caused by unusual or awkward social interactions between individuals. Conversations between a boss and an employee, conversations between friends, and public speaking are among the situations which are stressful for many people. Appropriate conduct during the interaction and correctly chosen words at the right time help smoothen the situation and relieve stresses that arises.

Existing techniques for stress management are deficient in several ways. While current technologies might implement biometric data tracking to detect stressful situations in a user's life, they do not suggest customized resources based on the specific experiences including customized counselor selection based on the level and cause of stress and anxiety. Stressful events are also operable to occur at any time, often without notice, impacting the mental health of the user and leading to possible depression.

Long-term stress has been linked to multiple health issues including, but not limited to, cardiac events, high-blood pressure, obesity, etc. Therefore, reducing stress is likely to allow people to live happier, healthier, and longer lives. Considering much of the stress experienced by individuals is rooted in social interactions, there exists a need to provide individuals with a platform to both monitor and lower the amount of stress experienced by the user.

None of the prior art discloses the use of Artificial Intelligence/Machine Learning (AI/ML) models to predict possible stressful situations before they occur and to proactively provide resources to prepare for such situations. Furthermore, none of the prior art discloses a platform operable to automatically link a user account to an online environment of other user accounts whose corresponding users either are experiencing or have experienced situations similar to the stressful situation of the user. Finally, none of the prior art discloses automatically providing a user with recommendations specific to a particular situation and user account to aid in decreasing stress in stressful situations.

In one embodiment, the present invention includes a system that helps users to communicate appropriately in different situations and events. In the event a user finds himself stuck in between awkward, tense, and embarrassing situations, proper communication and a correct attitude help the user persist in such situations effectively. The system provides the user with conversation scripts in form of modules to navigate such situations. The modules are operable to include resources such as blogs, Q&A, simulations, podcasts, etc. that pertain to the situation. These modules are further operable to simulate real-life scenarios and interactions for the users to boost their confidence while communicating.

The present invention includes a platform operable to measure stress within users' lives through biometric measurements using wearable and/or health monitoring devices. In one embodiment, the platform includes a server platform including one or more servers connected to an application on a mobile device including a processor and a memory such as a smartphone, wearable, or cellular phone. In one embodiment, the server platform is a cloud-based platform or a platform including one or more edge devices. As used herein, the term “platform” refers to the server platform and/or the application on a mobile device individually or in combination. The application identifies stressful events based on elevation in biometric measurements such as heart rate, blood pressure, sleep, indicators of anxiety, indicators of moods, and/or perspiration. Utilizing AI, the application is operable to determine resources that are most likely to relate to events and/or situations where the platform senses increased stress levels. The application is operable to then curate suggested conversation models and/or modules and relevant support resources for the user account. If the user needs additional support, the application also provides a connection with a counselor. A connection with the counselor is operable to be provided directly through the platform (i.e. through video chat, voice chat, etc.) and/or by providing contact information for a recommended counselor. Over time, the application aids users in acquiring the skills and confidence necessary to manage stressful situations, allowing users to achieve lower overall stress levels. Research shows positive outcomes from lower stress, benefitting users in many ways including, but not limited to, mental and physical health. In one embodiment, the counselor is operable to include a therapist.

The present invention is further operable to implement AI/ML via an analytics engine including an AI and/or ML engine. In one embodiment, the analytics engine is the analytics engine on the remote server. By way of example, the platform is operable to receive phrases and questions such as “How do I handle an abusive boss?”, “My significant other is cheating on me”, etc. and based on ML, natural language process (NLP), and/or sentiment analysis, determine how the phrases and questions align with resources operable to be presented by the platform. In one embodiment, this is achieved by the platform by assigning a confidence score to resources stored in at least one database based on the phrase and/or question received by the platform and past feedback provided to the system. The resources with the highest confidence scores represent resources that are most likely to be found useful in handling the situation provided to the platform. These resources are then operable to be presented by the platform if the resources have a confidence score over a threshold confidence score. In one embodiment, only the resources with the highest confidence scores is presented by the platform, such as the resources with the highest three scores, highest five scores, etc. The AI coupled with stress management functionality is further operable to predict and develop recommendations for stressful situations in real-time.

Referring now to the drawings in general, the illustrations are for the purpose of describing one or more preferred embodiments of the invention and are not intended to limit the invention thereto.

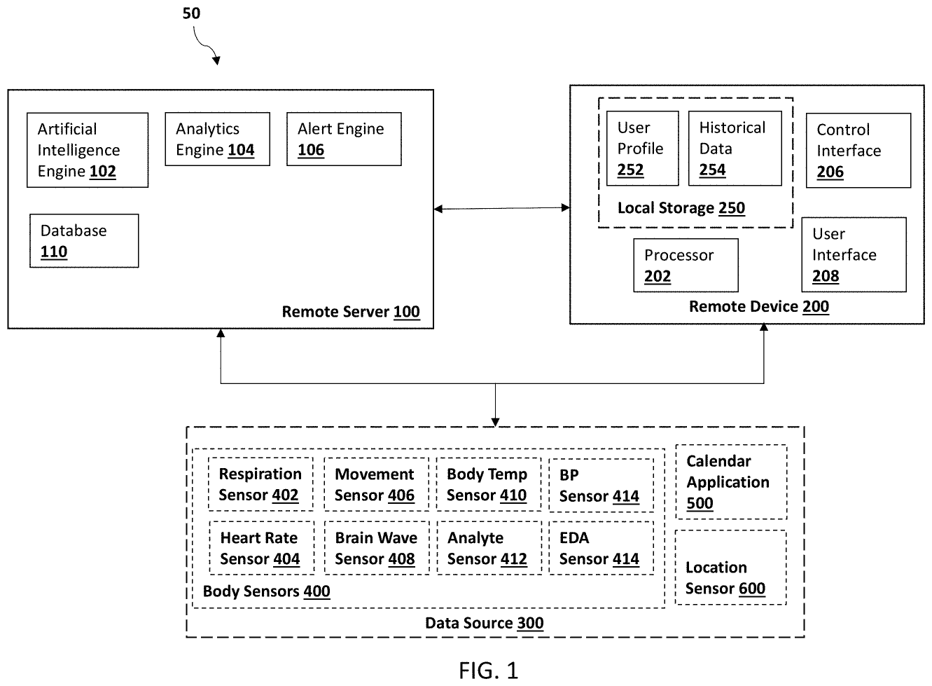

FIG. 1 is a block diagram of one embodiment of the stress management system 50. The stress management system 50 includes at least one remote server 100 and at least one electronic device 200 in network communication with each other and operable to receive data from at least one data source 300. The electronic device 200 is preferably remote from the at least one remote server 100. The stress management system 50 is configured to collect, process, analyze, model, and present data and/or insights related to the data collected by the at least one data source 300. The stress management system 50 is configured to process real-time or near-real-time data collected by the at least one data source 300.

The at least one remote server 100 includes a plurality of engines, at least one processor, and at least one memory. The plurality of engines includes, but is not limited to, a artificial intelligence engine 102, an analytics engine 104, and/or an alert engine 106. Preferably, each engine is operable to receive data from at least one other engine of the plurality of engines. For example, and not limitation, the analytics engine is configured to receive data from all of the other engines of the plurality of engines to provide an analysis of every aspect of the data. In one embodiment, the at least one remote server 100 is a cloud-based server or an edge device. The at least one remote server 100 preferably includes at least one database 110 operable to store information including, but not limited to, information related to at least one user account and/or the at least one data source, user account population data (i.e. data of all the users) used to determine patterns in the data, location data of all stress events, etc.

In one embodiment, the at least one remote server is operable to include a backend for the platform built using .NET or .NET Framework, however alternative embodiments are operable to include servers with a backend built using other suitable programming languages. The backend is operable to facilitate logics for at least one application, process data obtained from the at least one data source, and complete other tasks necessary to provide functionality to the platform, Preferably, the artificial intelligence engine is located on a separate remote server from the remote server storing and executing the backend of the platform. Preferably, the artificial intelligence server is built using Python. However other programming languages operable to facilitate artificial intelligence are operable to be used. In such embodiments, the remote server hosting the backend of the platform and the artificial intelligence server are operable to be in communication through at least one application program interface.

The at least one electronic device 200 includes, but is not limited to, a smartphone, a tablet, a laptop computer, and/or a desktop computer. The at least one electronic device 200 includes at least one processor 202, a control interface 206, a user interface 208 (e.g., a graphical user interface (GUI)), and local storage 250 (e.g., at least one memory). The at least one server 100 is operable to transmit and receive data from the at least one electronic device 200. In a preferred embodiment, the at least one electronic device 200 is in communication with the at least one data source 300. In such embodiments, data from the data source 300 is operable to be collected by the electronic device 200 where it is operable to be formatted and sent to the at least one server 100. Additionally or alternatively, the at least one remote server 100 is operable to receive data directly from the at least one data source 300. The at least one electronic device 200 is operable to store data in the local storage 250. In one embodiment, the at least one electronic device 200 further includes a speaker for playing audio. Through the user interface 208, the at least one electronic device 200 is operable to provide a user account access to a stress management platform.

The local storage 250 on the at least one electronic device 200 preferably includes a user profile 252. The user profile 252 stores user preferences and information including, but not limited to, user information (e.g., name, email address, phone number, address, contact information, profile picture, age, gender, etc.), information related to data sources associated with the user profile, information related to the data collected and processed by the system. Certain resources and functionalities are operable to be stored in the user profile that are operable to facilitate stress management in the user. As a non-limiting example, resources of particular interest to the user are operable to be downloaded directly to the user profile 252 for access when the remote server 100 is not in network communication with the electronic device. Additionally, data relating to the user profile 252 such as demographic and personal information is operable to be stored in the user profile 252. In one embodiment, the user account is further operable to store data relating to specific stress events and all the information captured with it including, but not limited to, biometric data including HRV delta, calendar data, location data, provided resources, data relating to at least one resolution, and/or user provided feedback.

The historical data 254 includes, but is not limited to, data relating to historical stress detected by the platform and/or data collected by and/or obtained from the data sources 300. Preferably, historical data is operable to include calendar data, correlating stress data, and metadata that provides context to the stress data. Historical data is further operable to include analytics data relating to the trends in stress levels associated with a user account. Furthermore, historical data is operable to include resources recommended and/or accessed by a user account and the effectiveness of such resources. Advantageously, this allows for the progress over time to be tracked, measured, and presented by the platform through the GUI. By way of example, the GUI is operable to display graphs showing heart rate, blood pressure, perspiration, sleep data, step count, anxiety, mood, and/or any other measurable biometric or physical attribute over time relevant to stress levels or emotional states over time periods around or during stressful events. Other historical data operable to be stored in the local storage include, but are not limited to, sentiment data, a journal of feelings associated with a user account, and text.

Data Sources

At least one data source is operable to be accessed by the system. Data sources include, but are not limited to, sources of data that collect, analyze, store, or process data that is insightful towards understanding of the stress associated with a user account.

In preferred embodiments, the at least one data source includes at least one body sensor. Body sensors include, but are not limited to, respiration sensors, heart rate sensors, movement sensors, brain wave sensors, body temperature sensors, analyte sensors, blood pressure (BP) sensors, electrodermal activity (EDA) sensors, and/or other sensors that collect biometric data. Data collected by these sensors is referred to collectively or individually as biometric data throughout the present application.

The respiration sensor measures a respiratory rate. In one embodiment, the respiration sensor is incorporated into a wearable device (e.g., a chest strap). In another embodiment, the respiration sensor is incorporated into a patch or a bandage. Alternatively, the respiratory rate is estimated from an electrocardiogram, a photoplethysmogram (e.g., a pulse oximeter), and/or an accelerometer. In yet another embodiment, the respiratory sensor uses a non-contact motion biomotion sensor to monitor respiration.

The heart rate sensor is preferably incorporated into a wearable device (e.g., Fitbit®, Jawbone®). Alternatively, the heart rate sensor is attached to the user with a chest strap. In another embodiment, the heart rate sensor is incorporated into a patch or a bandage. In further embodiments, the heart rate is determined using electrocardiography, pulse oximetry, ballistocardiography, or seismocardiography. In one embodiment, the heart rate sensor measures heart rate variability (HRV). HRV is a measurement of the variation in time intervals between heartbeats. A high HRV measurement is indicative of less stress, while a low HRV measurement is indicative of more stress. Studies have linked abnormalities in HRV to diseases where stress is a factor (e.g., diabetes, depression, congestive heart failure). In one embodiment, a Poincaré plot is generated to display HRV on a device such as a smartphone.

The movement sensor is an accelerometer and/or a gyroscope. In one embodiment, the accelerometer and/or the gyroscope are incorporated into a wearable device (e.g., Fitbit®, Jawbone®, actigraph). In another embodiment, the accelerometer and/or the gyroscope are incorporated into the electronic device. In alternative embodiments, the movement sensor is a non-contact sensor. In one embodiment, the movement sensor is at least one piezoelectric sensor. In another embodiment, the movement sensor is a pyroelectric infrared sensor (i.e., a “passive” infrared sensor). Alternatively, the movement sensor is incorporated into a smart fabric.

The brain wave sensor is preferably an electroencephalogram (EEG) with at least one channel. In a preferred embodiment, the EEG has at least two channels. Multiple channels provide higher resolution data. The frequencies in EEG data indicate particular brain states. The brain wave sensor is preferably operable to detect delta, theta, alpha, beta, and gamma frequencies. In another embodiment, the brain wave sensor is operable to identify emotion metrics, including focus, stress, excitement, relaxation, interest, and/or engagement.

The body temperature sensor measures core body temperature and/or skin temperature. The body temperature sensor is a thermistor, an infrared sensor, or thermal flux sensor. In one embodiment, the body temperature sensor is incorporated into an armband or a wristband. In another embodiment, the body temperature sensor is incorporated into a patch or a bandage. In yet another embodiment, the body temperature sensor is an ingestible core body temperature sensor (e.g., CorTemp®). The body temperature sensor is preferably wireless.

The analyte sensor monitors levels of an analyte in blood, sweat, or interstitial fluid. In one embodiment, the analyte is an electrolyte, a small molecule (molecular weight<900 Daltons), a protein (e.g., C-reactive protein), and/or a metabolite. In another embodiment, the analyte is glucose, lactate, glutamate, oxygen, sodium, chloride, potassium, calcium, ammonium, copper, magnesium, iron, zinc, creatinine, uric acid, oxalic acid, urea, ethanol, an amino acid, a hormone (e.g., cortisol, melatonin), a steroid, a neurotransmitter, a catecholamine, a cytokine, and/or an interleukin (e.g., IL-6). The analyte sensor is preferably non-invasive. Alternatively, the analyte sensor is minimally invasive or implanted. In one embodiment, the analyte sensor is incorporated into a wearable device. Alternatively, the analyte sensor is incorporated into a patch or a bandage.

The blood pressure (BP) sensor is a sphygmomanometer. The sphygmomanometer is preferably wireless. Alternatively, the blood pressure sensor estimates the blood pressure without an inflatable cuff (e.g., Salu™ Pulse+). In one embodiment, the blood pressure sensor is incorporated into a wearable device.

The electrodermal activity sensor measures sympathetic nervous system activity. In one embodiment, the electrodermal activity sensor is incorporated into a wearable device. Alternatively, the electrodermal activity sensor is incorporated into a patch or a bandage.

Further data operable to be collected by the at least one body sensor includes, but is not limited to, steps taken in a day, elevation change data (i.e. floors of stairs climbed), calories burned, amount of time spent in certain heart rate zones, resting heart rate, time spent in a mindful state, etc.

Advantageously, the present invention is operable to access further data sources as a means to gain more information regarding stress patterns. In one embodiment, data sources are operable to include at least one calendar-keeping software and/or applications such as, but not limited to, GOOGLE CALENDAR™, MICROSOFT OUTLOOK™, APPLE CALENDAR, SALESFORCE CALENDAR, etc. This data is referred to as calendar data throughout the present application. The calendar data collected by the system is operable to provide context regarding detected stress levels. For example, but not limitation, calendar data is operable to be analyzed to determine certain situations where stress is experienced and assist the platform in providing resources specific to that situation. In one embodiment, calendar data provided to the system is operable to allow the platform to identify attendees to certain events, recurring meetings in the calendar, days wherein the system senses more stress, etc. Based on the identification the platform is operable to issue notifications regarding upcoming events wherein certain attendees and/or situations arising at such event have previously caused the system to sense increased stress levels. The platform is operable to collect and provide resources that are useful in overcoming stressors at said events. In one embodiment, the platform is further operable to identify a certain attendee as a stressor and tag events including that attendee as possible stressors.

Additional data sources further include location data collected by one or more location sensors. Location data is operable to be collected through a variety of methods including, but not limited to, global positioning systems (GPS), geofencing, triangulation, etc. Location data is operable to be collected by a variety of sensors used to track the location of devices associated with a user account. Location sensors are operable to be included in the electronic device and/or a wearable worn by the user. Advantageously, the collection of calendar data and/or location data allows for the system to provide a customized context for a user account regarding specific stressors as a means to determine the reasoning and context for stressors. Also, this allows for the system to provide optimal recommendations of resources for the stressors based on the specific scenarios that cause stress for the user. This data is referred to as location data throughout the present application.

In one embodiment, data sources are further operable to include other third-party data sources. Third-party data sources include, but are not limited to, online sources such as external applications, web sites, databases, etc. In one embodiment, one or more application program interfaces (APIs) are operable to be used by the system to interface and pull data from a third-party source. Exemplary third-party data sources are operable to include third-party systems that collect and analyze sensor data and/or calendar data. Third party systems include, but are not limited to, Fitbit®, Jawbone®, Amazon® Alexa®, Outlook®, and/or Apple® Health. Alternatively, the system is operable to include web crawlers, database crawlers, and/or crawlers designed to crawl other sources of data. In one embodiment, a combination of APIs and crawlers are used to obtain as much data as available to the system, and to update the data in real time or near real time, or at predetermined intervals (e.g., hourly, daily, weekly, monthly, etc.) In one embodiment, the web crawler is used to obtain data calendar data and/or biometric data from websites. However, the web crawler and any other crawlers are operable to collect other data such as, but not limited to, data specific to the user account that is helpful to determining the stress level of the user and/or providing context to the stress.

Additionally or alternatively, the platform is operable to collect data directly from the electronic device using sensors in the electronic device. Examples of sensors in the electronic device operable to provide data to the system include, but are not limited to, audio sensors (i.e., microphones), location sensors, image/video sensors (i.e. cameras), heartrate sensors, and/or any other sensor operable to be included in the electronic device. This data is further operable to be utilized in the determination of resources provided to the user account.

Additionally, the platform is operable to collect data relating to the sentiment of the user. In one embodiment, data relating to the sentiment of the user is operable to be determined by prompting the user account with questions such as, but not limited to, “how are you feeling today?” and analyzing the answer input by the user to determine a mood of the user. In one embodiment, answers incorporating language typically associated with negative connotations are used to identify a bad mood, a sad mood, or any other negative mood. In a preferred embodiment, the input is analyzed by implementing sentiment analysis techniques. Further, data used to determine sentiment data is operable to include a journal of thoughts maintained by the user. In such embodiments, sentiment analysis is performed on entries into the journal of thought and used to determine at least one likely sentiment of the user.

Application

The stress management platform preferably provides a graphical user interface (GUI) operable for user interaction through an electronic device.

In one embodiment, the stress management platform includes a mobile application. In one embodiment, the mobile application is a native mobile application. Native mobile applications are built for platforms with the platform Software Development Kit (SDK), tools, and languages, typically provided by the platform vendor. Examples include, but are not limited to, xCode/Objective-C for iOS, Eclipse/Java for Android, Visual Studio/C# for Windows Phones, and FLUTTER for cross-platform functionalities. In another embodiment, the mobile application is a mobile web (Mobile Web) application. Mobile Web applications are server-side applications, built with any server-side technology including, but not limited to, PRP, Node.js, and ASP.NET, that render Hypertext Markup Language (HTML) that has been styled so that it renders well on a device form factor.

In another embodiment, the stress management platform includes a hybrid mobile application (Hybrid App). A Hybrid App is an application that is written with the same technology used for web sites and mobile web implementations, and that is hosted or runs inside a native container on a mobile device. Hybrid Apps use a web view control to present the HTML and JAVASCRIPT files in a full-screen format, using the native browser rendering engine and not the browser itself. For example, WEBKIT is the browser rendering engine that is used on iOS, ANDROID, BLACKBERRY, SAFARI, MAIL, APP STORE, and others. This means that the HTML and JAVASCRIPT used to construct a Hybrid App are rendered or processed by the WEBKIT rendering engine and displayed in a full-screen web view control, not in a browser. In addition, Hybrid App implements an abstraction layer that exposes the device capabilities, native Application Programming Interfaces (APIs), to the Hybrid App as a JAVASCRIPT API. This is not possible with Mobile Web application implementations because of the security boundary between the browser and the device APIs. Through this abstraction layer, a common set of APIs are exposed in JAVASCRIPT, and these JAVASCRIPT APIs work on any device supported by the framework.

In one embodiment, the browser rendering engine is further operable to be GECKO, GOANNA, KHTM, PRESTO, TASMAN, TRIDENT, BLINK, SERVO, and/or EDGEHTML. In another embodiment, the application includes both a remote application and a desktop application.

The present invention is operable to provide applications including, but not limited to, a base application and/or an SDK for custom applications. The SDK for custom applications enables functionality including, but not limited to, registration, selling, support, maintenance, service, education, and/or marketing.

The application is operable to collect, process, analyze, and transmit data relating to stress levels associated with a user account and allow access to the stress management platform through the GUI.

Authentication

For sensitive applications, adding a second authentication method and/or factor is appropriate (also known as “two-factor authentication”). In one embodiment, two-factor authentication (2FA) functionality is enabled via receipt of input through the GUI. This includes applications that provide access to sensitive information (e.g., credit card numbers, health records) or allow the transfer of funds. Mobile applications use HyperText Transfer Protocol (HTTP) as the transport layer. The HTTP protocol itself is stateless, so there must be a way to associate a user's subsequent HTTP requests with that user, otherwise, the user's log in credentials need to be sent with every request. In one embodiment, the second authentication method is stateful authentication. Stateful authentication generates a unique session ID when the user logs in. In subsequent requests, this session ID serves as a reference to the user details stored on the server. The session ID is opaque, in that it does not contain any user data. In one embodiment, the session ID is random. In one embodiment, the second authentication method is stateless authentication. With stateless authentication, all user-identifying information is stored in a client-side token. The token is passed to any server or micro service, eliminating the need to maintain a session state on the server. Stateless authentication is often factored out to an authorization server, which produces, signs, and encrypts the token upon user login.

In one embodiment, the second authentication method is an additional user password, wherein the additional user password is distinct from the user's original account password. In one embodiment, the second authentication method is a PIN number. In another embodiment, the second authentication method is a user-created pattern on a mobile computing device. In yet another embodiment, the second authentication method is a one-time password generator. In yet another embodiment, the second authentication method is a hardware token generating a one-time password. In yet another embodiment, the second authentication method is user biometric data. User biometric data includes, but is not limited to, a fingerprint, a retinal scan, a haptic vein scan, facial recognition, voice recognition, and/or ear recognition. In one embodiment, the second authentication method is a passive contextual authentication. Passivate contextual authentication includes, but is not limited to, geolocation, IP address, time of day, and/or the device being used by the user (e.g., MAC address).

In one embodiment, the second authentication method uses the OWASP Mobile AppSpec Verification Standards (MASVS). MASVS is split into two authentication levels. In one embodiment, the MASVS level is level one. Level one MASVS functions with non-critical applications and suggests the following authentication requirements: if the app provides users with access to a remote service, an acceptable form of authentication such as username and/or password authentication is performed at the remote endpoint; a password policy exists and is enforced at the remote endpoint; the remote endpoint implements an exponential back-off, or temporarily locks the user account when incorrect authentication credentials are submitted an excessive number of times.

In one embodiment, the MASVS level is level two. Level two MASVS functions with sensitive applications and includes the following, in addition to the level one requirements: a second factor of authentication exists at the remote endpoint and the second factor authentication requirement is consistently enforced; step-up authentication is required to enable actions that deal with sensitive data and/or transactions; and the application informs the user of the recent activities with their account when they log in.

In another embodiment, single sign-on (SSO) functionality is enabled (e.g., via user input). SSO is a property of access control of multiple related, yet independent software systems. With this property, a user logs in with a single ID and password to gain access to any of several systems. In one embodiment, SSO uses a Lightweight Directory Access Protocol (LDAP) and stored LDAP databases on servers. In another embodiment, SSO uses cookies. In one embodiment, SSO is Kerberos-based, wherein the initial sign-on prompts the user for credentials and gets a Kerberos ticket-granting Ticket (TGT). In one embodiment, SSO is smart-card-based, wherein initial sign-on prompts the user for the smart card. Additional software applications also use the smart card, without prompting the user to re-enter credentials. Smart-card-based SSO is operable to use certificates and/or passwords stored on the smart card. In one embodiment, SSO is based on a Security Assertion Markup Language (SAML). SAML is an XML-based method for exchanging user security information between an SAML identity provider and a SAML service provider.

In a preferred embodiment, the platform is operable to meet all security standards included in the OWASP Web Security Testing Guide Version 4.2 Published Dec. 3, 2020 by the OWASP and/or the most recent version of the OWASP Web Security Testing Guide each of which is incorporated herein by reference in its entirety.

Account Creation and Data Linkages

User account information is operable for editing after the account is created. In addition, the platform is operable to provide an account dashboard (e.g., via the GUI). The account dashboard is operable to display information associated with the user's account including, but not limited to, past and current stress data, resources for stress management, a chatbot for stress management, user account settings, user reviews, and/or shortcuts to the user's dashboard. In the present disclosure, data including audio data and/or text data that is operable to be interpreted by the chatbot is referred to generally as chat data.

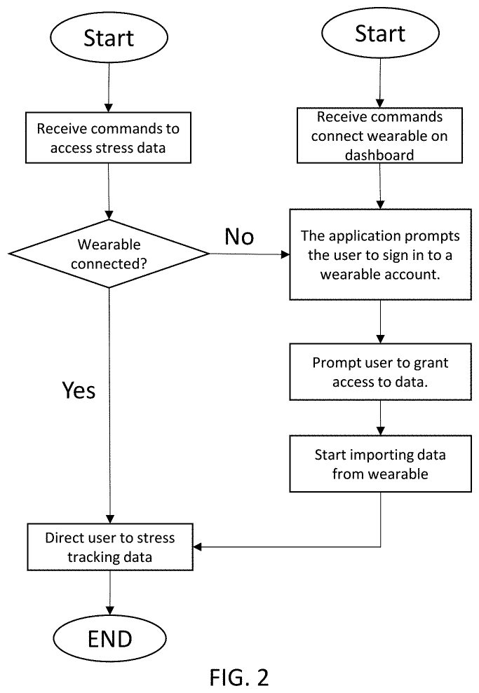

After an account is created, data sources are operable to be linked with the electronic device and/or the remote server. FIG. 2 illustrates a method for connecting a body sensor data source to the platform according to one embodiment of the present invention. In the decision tree shown in FIG. 2, the application provides an option for a user to connect a wearable via the GUI. Upon selection of the option to connect the wearable through the GUI, the application redirects to a third-party login page associated with the wearable. Upon successful login, the application prompts the user account to grant the platform access to a specific data type collected by the wearable. Upon the platform being granted access, past data collected by the wearable and future data collected by the wearable is automatically imported into the application. In one embodiment, this is achieved by accessing the data collected by the wearable through at least one API that interfaces with at least one application and/or database associated with at least one wearable. In an alternative embodiment, upon being granted access, the application is operable to collect data directly from the wearable. This is operable to be achieved by configuring the wearable with instructions to communicate directly with the stress management application.

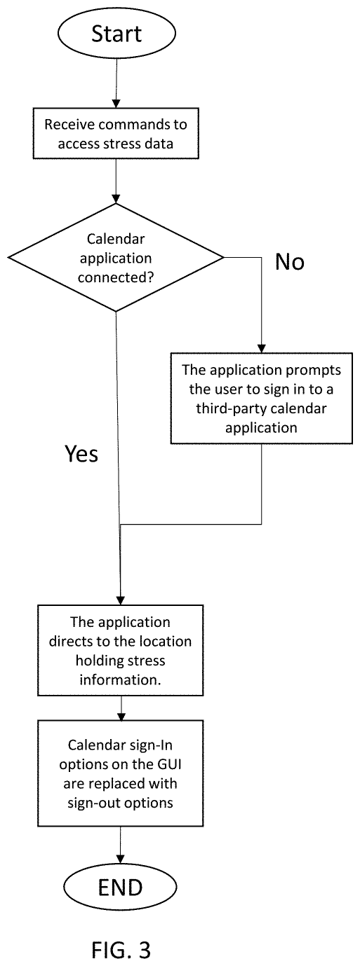

FIG. 3 illustrates a method for connecting a calendar application data source to the platform according to one embodiment of the present invention. In the decision tree, the application provides an option to connect a calendar application. Upon navigation to a location holding stress information, the application checks to determine if a third-party calendar application has been connected. If not, the application prompts the user account to sign in to a third-party calendar application. Upon successful sign-in to the third-party calendar application, the application redirects back to the location holding stress information. Any options on the GUI that directed to the calendar sign-in page are then replaced with options that allow the user to disconnect the calendar application. Once connected, the application and/or stress management platform is operable to retrieve calendar data from the calendar applications through an API.

In one embodiment, an existing social media account is linked to the stress management platform. Examples of social media accounts operable to be linked with the platform include, but are not limited to, FACEBOOK, TWITTER, INSTAGRAM, SNAPCHAT, LINKEDIN, TUMBLR, PINTEREST, SINA WEIBO, REDDIT, TIKTOK, VKONTAKTE, FLICKR, MEETUP, INTERNATIONS, XING, and/or NEXTDOOR. Alternatively or additionally, other third-party sources and accounts are operable to be linked to the stress management platform.

In one embodiment, the platform includes an internal social media platform for the internal community to interact and communicate. Using the internal social media platform, posts from the community are operable to viewed by other members of the community. In one embodiment, user accounts are operable to follow other user accounts and/or topics of interest.

Account Types

In some embodiments, the platform includes more than one user account type. Examples of such account types include, but are not limited to: an administrator, an employee, a parent, a child, a teacher, a student, a general user, etc. In one embodiment, the platform provides different permissions based on the account type. In a non-limiting example, a parent and a child create accounts on the application. The parent is listed as a parent to the child and is permitted full access to the child account. The parent account is operable to track and collect data on stress associated with the child account and the parent account. In a further non-limiting example, a child account is not able to access stress relieving resources of a specific class unless a parent account grants specific permissions to the child when making the child account or enters credentials of the parent account to provide permission to the child account. In one embodiment, a parent account is operable to lock certain features from being accessed by the child account to prevent the child account from accessing resources that are deemed inappropriate for the child's age by the parent account. In one embodiment, a parent account is operable to access the stress data of at least one child account. The platform is further operable to provide the parent account with resources based on the stress data of the child account. In a non-limiting example, the platform collects stress data from the child account indication stress during an interaction with the owner of the parent account. Resources are then operable to be provided by the platform to the parent account to communicate in ways that are helpful in lowering the stress levels of the child. Advantageously, this allows the parent account to access resources that improve communication between the child and the parent and improves the stress levels of both parties. Similar permission rules are operable to be enforced for hierarchal relationships other than parent-child including, but not limited to, administrator-employee, teacher-student, etc.

Permission rules are preferably designated for each associated account and are operable to be different for similar account types. For example, an administrator account is associated with a first employee account, a second employee account, and a third employee account having a first set of permissions, a second set of permissions, and a third set of permissions, respectively. The first set of permissions is not equivalent to the second set of permissions, and the second set of permissions is not equivalent to the third set of permissions. Alternatively or additionally, accounts are operable to be designated as admin accounts and user accounts. In such embodiments, the admin accounts are operable to upload new content to the platform, while the user accounts are only allowed to access the content.

Conversation Models and Resources

In some embodiments, the platform is operable to retrieve one or more conversation models based on analysis of the one or more stressor events. The one or more conversation models are operable to include conversation scripts relevant to at least one situation the user finds stressful. The one or more conversation models are operable to include a sequence of dialogues and tips to include the dialogue in conversations. The platform is further operable to retrieve additional resources associated with the one or more conversation models. Resources and conversation models are operable to be stored in the database of the at least one remote server and transmitted to the one or more electronic devices. Conversation models and resources provided to the electronic device, advantageously, offer useful insights and information regarding ways to successfully overcome the stressful situations. Additionally, or alternatively, embodiments of the one or more conversation models include, but are not limited to, speeches that are advantageous to improving the situation and/or dialogues suitable for aiding the user to settle conflict regarding stressful situations. Although the conversation models are illustrated as text in a graphical user interface (GUI), the conversation models are operable to include audio and/or video content. In one embodiment, the text displayed in the GUI is operable to be played as audio output through a speaker of the electronic device, and a microphone of the electronic device is operable to receive audio input in response to the audio output. In another embodiment, the text shown in the GUI is operable to be part of an interactive video played by the electronic device represented in either textual or audio form, with the GUI operable to receive input through the interactive video via a speaker or through touch screen input. In one embodiment, the electronic device includes a camera operable to capture video and/or image data from a user, including facial expressions, with the platform operable to analyze facial expressions in real time or near real time and provide real time or near real time feedback or information based on the analyzed facial expressions. In additional embodiments, the electronic device includes virtual reality (VR) components operable to receive input from a user and update the virtual reality environment based on those inputs. Examples of VR components include VR headsets or other wearable VR accessories or devices. By way of example and not limitation, the VR headset in one embodiment includes the VR headset described in U.S. Pat. No. 10,261,579, which is incorporated herein by reference in its entirety.

Advantageously, the resources provided help to build confidence in the user's abilities to handle situations in which they find themselves stressed, reducing overall stress levels. Additional or alternative resources include, but are not limited to, blogs, simulations, podcasts, etc. that relate to at least one stressful situation detected by the platform. Additional or alternative resources are also operable to be stored in the database of the at least one server and delivered to the electronic device upon conditions deeming them necessary. In one embodiment, simulations include simulation audio and/or video content relating to diversity, equity, and inclusion (DE&I) topics and workplace anti-discrimination topics such as addressing or preventing racial bias, addressing or preventing gender discrimination, addressing or preventing religious discrimination, addressing or preventing age bias, addressing or preventing honest feedback avoidance to people of color, addressing or preventing microaggressions, addressing or preventing disability discrimination, addressing or preventing pregnancy discrimination, addressing or preventing parental status discrimination, addressing or preventing sexual orientation discrimination, addressing or preventing national origin discrimination, addressing or preventing sexual harassment, and/or addressing or preventing retaliation discrimination. DE&I and anti-discrimination topics are also operable to be presented as textual content, video content, audio content, image content, or any combination thereof.

FIG. 3 illustrates a method for managing stress experienced during events according to one embodiment of the present invention. The method includes a step of receiving data associated with at least one user account from at least one electronic device. The at least one electronic device is operable to detect and/or access biometric data. Preferably, the biometric data is generated by at least one wearable worn by the user. Data regarding the situation is operable to be accessed from a variety of data sources including, but not limited to, the calendar application, data collected by the electronic device, location data, etc.

In some embodiments, the at least one remote server is configured for receiving one or more conversations from chat data provided by the one or more electronic device. The conversations are operable to be conversations submitted by a user account relating to stressors for which resources are being requested. Advantageously, in such embodiments, the platform is supplied data relating specifically to the subject causing stress, allowing for highly relevant resources to be provided. Preferably, conversations are provided to the system as audio data and/or text data, further operable to be referred to as chat data, that are operable to be processed and analyzed by natural language processing (NLP).

Preferably, the remote server is configured for receiving feedback from one or more electronic devices. Such embodiments allow the platform to improve the relevancy and effectiveness of the models and resources provided. As a non-limiting example, the platform provides resources to a user account that do not directly relate to the stressor of the user. The platform is operable to receive feedback regarding the irrelevancy of the resource and/or models provided. This data is passed into the AI engine and/or the ML engine of the platform so the platform will provide different resources and/or models in the future. In one embodiment, this is performed by receiving the data relating the feedback to the learning engine. Alternatively, or additionally, the AI engine and/or the ML engine is operable to be included in an analytics engine external to the remote server and accessed by the remote server through an API. Further embodiments are operable to provide electronic devices with mindfulness exercises to assist in stress reduction.

In one embodiment, the platform is configured for analyzing the one or more conversations. The platform is therefore operable to determine one or more conversation models based on the analysis of the one or more conversations presented to the system. In one embodiment, the one or more conversation models include conversation scripts. Additionally or alternatively, the one or more conversation models include a sequence of dialogues and a way to include the dialogues in conversations. In one embodiment, the platform is configured to determine the one or more mindfulness techniques based on the feedback. Advantageously, the one or more mindfulness techniques calm the user and deduce an appropriate reaction for the environment. Conversations and mindfulness techniques are both preferably stored in the storage of the at least one remote server.

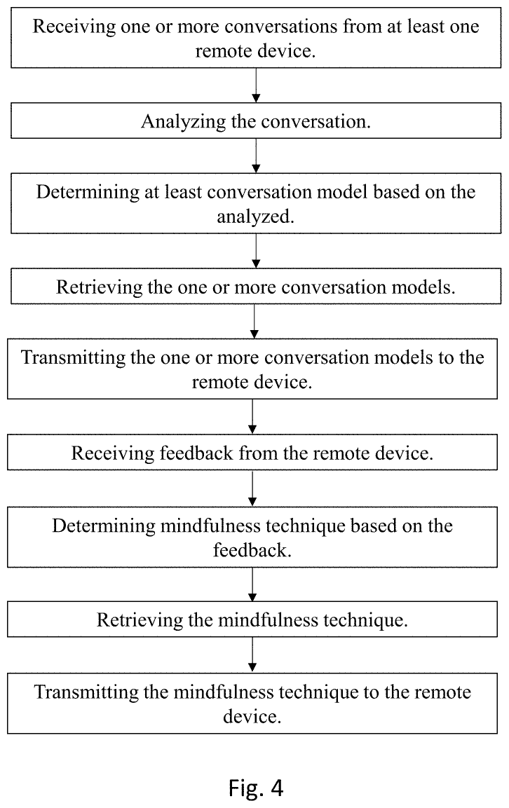

FIG. 4 is a flowchart of a method for managing stress based on conversations. The method includes a step of receiving one or more conversations from one or more electronic devices. The one or more conversations are operable be associated with and assigned to one or more user accounts. In one embodiment, the platform senses that one or more conversation participants are facing challenges during conversations in an environment. The platform is operable to detect that one or more conversation participants are operable to feel stressed and anxious during the conversations through stress level determination based on data received by the platform. In one embodiment, the method includes a step of analyzing the one or more conversations and recommending resources and models based on the processed conversation data.

In another embodiment, the platform is operable to provide counselor information to the one or more electronic devices. The platform is further operable to receive feedback regarding the effectiveness of the counselor information. By receiving feedback the platform is operable to determine which counselors are most appropriate for certain situations by analyzing the feedback. Counselor information associated with the appropriate counselor is operable to be stored in the databases of the at least one remote server and transmitted to the at least one electronic device. The counselor information is operable to include, but is not limited to, details including name, contact information, advice, etc. Although the term “counselor” is used throughout the present specification, one of ordinary skill in the art will understand that the platform of the present invention is also operable to provide therapist information and recommendations through a therapist network.

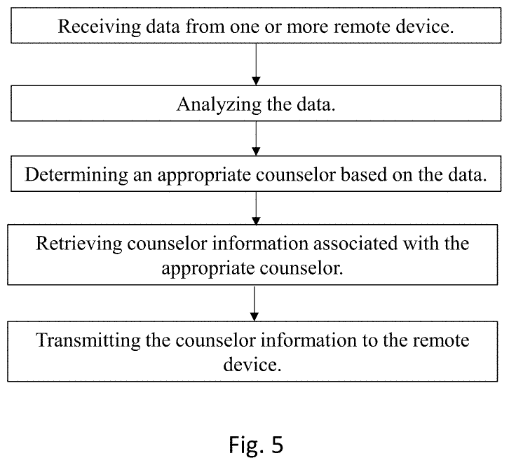

FIG. 5 illustrates a method for recommending counselors based on user feedback according to one embodiment of the present invention. The method includes a step of receiving feedback from one or more electronic devices, which are operable to be associated with one or more users. In the event the platform offers a counselor to the electronic device that the user finds ineffective, the platform is operable to receive feedback about its ineffectiveness. This data is then used to train the artificial intelligence engine or the machine learning engine utilized by the platform to recommend more effective counselors in future situations.

In one embodiment, conversation models operable to be displayed through the GUI include, but are not limited to, models directed towards giving feedback, asking for feedback, receiving feedback, confrontational conversation, working in a team, coaching, delegating responsibilities, accountability, etc. Further, certain conversation models are operable to be split into more than one part. In such embodiments, one part of the conversation model is operable to provide prompts to aid in planning for a certain type of conversation while another part of the conversation model is operable to provide prompt to aid in carrying out the conversation.

Further, resources operable to be provided to the user account in response to high stress situations include self-talk methods or methods where resilience and adaptability are implemented in an attempt to aid the user come to a point of self-realization about events. In such embodiments, resolution does not include confrontation or even participation from another individual. Advantageously, such methods are operable to assist a user in decreasing overall stress levels by simple reflection.

In a preferred embodiment, conversation models are operable to be presented to a user account through a chatbot. Advantageously, this allows for the conversation model to be dynamic and change based on input from the user. As a non-limiting example, the platform receives input from a user that is counterproductive to conflict resolution. The platform is operable to adapt its conversation model based on the input received.

In some embodiments, resources operable to be provided to the user account to assist with handling stressful situations further include a blog recommendation, a podcast, and/or a video. In such embodiments, these resources are operable to be specific to situations experienced by the user similar to conversation models. As a non-limiting example, the platform determines that a user is experiencing stress while the user is working with a team. In such a scenario, the platform is operable to provide the user account with blogs with members experiencing similar stressful situations as well as a podcast and/or video providing strategies to communicate and work more efficiently in a team setting.

The following is an example of a conversation model relating to a conversation with a team. In the example, questions are presented and the platform is operable to receive input to the questions.

Beach Ball Preparation Form

1) The issue is

Be concise. In 1 or 2 sentences, get to the heart of the issue. Is it a concern, challenge, opportunity or recurring problem that is becoming more troublesome?

2) It's significant because

What's at stake? For example: how does this affect profitability, people, products, services, customers, timing, the future, or other relevant factors? What is the future impact if the issue is not solved?

3) My ideal outcome is

What specific results do I want?

4) Relevant background information

Summarize with bullet points: What, why, where, when, how, who, etc.; which forces are at work; what is the current status?

5) What have I done up to this point?

What have I done so far?

6) Options I am considering

What options am I considering? What am I leaning toward doing?

7) The help I want from the group is

What do I want from the group? For example: alternative solutions, confidence regarding the right decision, identification of consequences, where to find more information, critique of current plan, etc.

The following is an example of a conversation model relating to conducting a conversation with a team.

Conduct Conversation

Beginning the Conversation

1) Thank everyone for coming.

2) Give everyone a copy of the beach ball preparation form and talk through it to quickly focus attention and resources on the topic.

3) Tell your team members that you want to hear their perspectives.

Remember, after the first three steps you move from being in presentation mode to learning mode. Now you are listening, being curious, drawing out the perspectives, opinions, and “stripes” of everyone else. In effect, you are saying I'm inviting you to influence me!

Continuing the Conversation

4) Make sure that you hear from each team member

Meeting Engagement Tips
Make sure to not discount or disprove anyone's idea.
Give internal processors time to speak up and encourage their thoughts.
When someone says, “I don't know” say “What would it be if you did know?”
Say, “Please say more about that” if someone's comment seems incomplete or hard to understand.”

Wrapping Up the Conversation

5) Ask each team member to write down a concise answer to this question: “What would you do if you were in my shoes?”

6) Have each person read his/her advice. Do not respond, except to say, “Thank you.”

7) After everyone has read their advice, tell them what you've heard and ask, “Did I miss anything essential?”

8) Thank everyone for their contributions and tell them what action you are prepared to take and when you will take it.

9) Ask them to sign their recommendations and give them to you so that you can follow up with them if you′d like more information.

10) Get back to them once you have made a decision or taken action. This follow up step is so important. It lets them know that you valued their time even if you did not take their recommendation. This helps people feel included.

The following is an example of a conversation model relating to coaching.

1) Identify the issue

“What is the most important thing you and I should be talking about today?”

Give them time to think about this. Remember, in the coaching conversation, it's the coachee who chooses the topic.

2) Clarify the issue

“What's going on? How long has it been going on? Am I understanding you correctly?

Is the issue ______?

Paraphrase—check your perception. Your job is to help the individual discover what this conversation wants and needs to be about.

3) Determine current impact

“How is this issue currently impacting you? Others? The company? What other results is this situation currently producing? (What else? What else? What else?)”

As you consider these results, what do you feel?

4) Determine future implications

“If nothing changes, what are the implications? What's likely to happen? What is at stake for you, for others, for the company if nothing changes? (What else? What else? What else?)”

When you consider these possible outcomes, what do you feel?

5) Examine personal contribution to the issue

“How have you contributed to this problem/issue? What piece of this issue has □our DNA on it?”

6) Describe the ideal outcome

“When this issue is resolved, what difference will that make? What results will you enjoy? Others? The company?”

When you contemplate these results, what do you feel?

7) Call to action

“What is the most important step you could take to move this issue towards resolution?”

When will you take it? What's going to try to get in your way and how will you get past it? When can you follow up with me?

The following is an example of a conversation model relating to delegating tasks. In the example, questions are presented and the platform is operable to receive input to the questions. Further this example uses the Root, Trunk, Branch, Leaf decision making model. This model teaches decisions are operable to be thought of with respect to the anatomy of a tree. Leaf decisions, like leaves of a tree are minor and fairly insignificant if they are wrong, similar to how a tree quickly grows back a leaf in the even they are plucked without much damage to the tree in whole. Branch decisions are bigger decisions that have more significant impact but still do not leave lasting damage, similar to cutting a branch off a tree. Trunk decisions are even larger decisions that, similar to the trunk of a tree, have the potential to cause gross harm to an organization, individual, company, etc. Root level decisions are decisions that are integral to the survival of the entity, similar to how roots are integral to the survival of a tree. More information about this model can be found in Scott, S. (2004). Fierce conversations: Achieving success at work & in life, one conversation at a time. New York: Berkley Books. which is incorporated by reference herein in its entirety.

What activity or responsibility (though it is important to the organization and you are good at it) is no longer the best use of your time?

To whom would you like to give this responsibility?

At what level? (Root, Trunk, Branch, Leaf)

By when?

The following is an example of a conversation model relating to alignment when delegating tasks. In the example, questions are presented and the platform is operable to receive input to the questions.

How do you feel about taking on this responsibility?

What do you think your next steps will be?

What roadblocks do you see trying to get in the way?

What assistance do you need from me?

What else?

The following is an example of a conversation model relating to confrontational conversations. In the example, the model is operable to receive input at the blanks demarked in the present disclosure with an “*” symbol.

60 Second Opening Statement

1) Name the issue

I want to talk with you about the effect *

Is having on *

2) The specific example that illustrates the behavior or the situation you want to change

For example(s), *

3) Describe your emotions around this issue

I feel *

4) Clarify why this is important—What is at stake to gain or lose for you, for others, for the team, or for the organization

From my perspective the stakes are high. * is at stake. And most importantly *, is at stake.

5) Identify your contribution(s) to this problem

I recognize my fingerprints, I have *. For this, I apologize.

6) Indicate your wish to resolve the issue.

I want to resolve this with you (restate the issue/the effect).

7) Invite your partner to respond

I sincerely want to understand your perspective. Talk with me.
During the Opening Statement, I am in presentation mode.
Now it's my turn to go into learning mode, into Mineral Rights mode (questions, questions, questions.)
Remember, I'm talking from my stripe or perspective; I'm not presenting it as THE truth. I need to know what your stripe or perspective is to discover the full truth.

The following is an example of a conversation model relating to conducting the previously described confrontational conversation.

8) The interaction phase. This is where the bulk of the conversation happens. You are in explorer mode, asking questions, interrogating reality and provoking learning. Tips for successful interaction include:
Use paraphrasing
Ask questions for clarification
Reaffirm understanding
Get curious—avoid jumping down rabbit holes. Stay with “help me understand . . . ”
Remain calm. Restate the issue statement to get back on track and refocused.
Resolution

9) What was learned? Where are we now? What is needed for resolution? What was left unsaid that needs saying? What is our new understanding? How can we move forward from here, given this new understanding?

10) Make a new agreement and have a method to hold each other accountable.

The following is an example of a conversation model relating to finding a waypoint for feedback. In the example, questions are presented and the platform is operable to receive input to the questions.

The Waypoint Model

The Waypoint Model, is simple and specific. It's made up of three important steps that are easy to remember: EXPERIENCE, EXPLORE, and EXPLAIN.
EXPERIENCE is the “when,” “where,” and “what.”
EXPLORE is having curiosity about the other person's perspective.
And, depending on the other person's perspective, EXPLAIN.

Experience Where? When? What?

What was YOUR experience of what happened? Be specific. As though you were watching a video.

Explore Get Curious

Ask: What's true for you? What was your experience? How do you see this? Can you tell me what was going on? I'm curious if you see what I see.

Explain Why It Matters

What are the results if this behavior continues? What is the impact if nothing changes? For them? For the team? For the organization?

The following is an example of a conversation model relating to asking for feedback. In the example, questions are presented and the platform is operable to receive input to the questions.

1) Experience

What is one of your desired results?

2) Explore

From whom do you need feedback along the way? Why that person?

3) Explain

What results are you currently getting and where can you improve?

4) Your Ask

Write out your “ask” in 2-3 sentences.

Example: When I lead a meeting, my goal is to have a robust, transparent conversation with everyone so that we understand and respect one another's perspective. (experience) I would appreciate hearing from you about ways in which I could have improved the experience and outcomes of the meeting. (explore) I ask because it's easy for me to assume the meeting is going well, but there is always room for improvement. (explain)

6) Timing

When will you have this conversation?

The following is an example of a conversation model relating to giving feedback. In the example, questions are presented and the platform is operable to receive input to the questions.

Give Feedback

Experience

1. Before you give feedback, ask yourself:
What is m□ true intention with giving feedback?
Am I making assumptions about this person and their abilities, decisions, behaviors?
Have I laid out my expectations in clear and direct terms?
Have I set realistic goals with this person?

Who

2. Who do you need to have a feedback conversation with? Who needs to have you hold a mirror up for them, to have a waypoint set on their behalf?

Experience Where? When? What?

3. What was YOUR experience of what happened? Be specific—as though you were watching a video.

Explore Get Curious

4. Ask: What's true for you? What was your experience? How do you see this? Can you tell me what was going on? I'm curious if you see what I see.

Explain (when it is required) Why it matters

5. What are the results if this behavior continues? What is the impact if nothing changes? For them? For the team? For the organization?

The following is an example of a conversation model relating to receiving feedback. In the example, questions are presented and the platform is operable to receive input to the questions.

Receive Feedback

To receive feedback in the most effective way possible, stay focused on the 4 Objectives of a Fierce Conversation:

1. How might we Interrogate Reality when we receive feedback? (We keep in mind that there are multiple perspectives, not only our own.)

2. How could we Provoke Learning when we receive feedback? (We choose curiosity over being “right;” we remain open to being changed through this conversation.)

3. How does Tackle Tough Challenges apply in receiving feedback? (We don't get defensive; we stay present to whatever it is we hear, knowing that it took courage for the other person to speak their truth.)

4. How could we enrich the Relationship when receiving feedback? (We stay focused on how our ability to receive feedback builds trust; the conversation IS the relationship.)

Experience Where? When? What?
Listen carefully to what is being said and how
Say “thank you”
Decide what you can learn from the feedback.

Explore Get curious
Be curious.
Ask for clarification and examples: Can you give me some specific examples? Can you say more about that? Where else have you seen me demonstrate this? How long have you been noticing? What has the impact been on you and me, on others, on the team?

Explain Why It Matters
Take responsibility for the impact and present your thoughts.
Reiterate gratitude for the feedback and share WHY it is important to you.
Articulate your future focus.

The following is an example of a conversation model relating to a context results cycle for maintaining accountability. In the example, questions are presented and the platform is operable to receive input to the questions.

Accountability

1) Context Results Cycle

In what ways might the stories I tell myself, be impacting my results?

2) Context

What is a context I hold today?

3) Assessment

What evidence have I gathered?

4) Emotions

What am I feeling as a result?

5) Behavior

What is showing up behaviorally?

6) Results

What results are being created?

The following is an example of a conversation model relating to conducting an accountability conversation. In the example, questions are presented and the platform is operable to receive input to the questions.

Idea 1

Our careers, our companies, our relationships and our very lives, succeed or fail, gradually then suddenly, one conversation at a time.

Highly effective people track their trends—they pay attention to each conversation to see if they are moving closer to, or further away from their desired “outcome”

Application suggestion—“what is a conversation that you have been putting off? What is this costing you?”

Idea 2

The conversation IS the relationship.

If we lower the standards about how often we talk, what we talk about, and most important, the degree of authenticity we bring to our conversations—gradually, then suddenly, the relationship suffers.

What should I START doing, STOP doing, and CONTINUE doing to improve both the conversation and the relationship?

Idea 3

All conversations are with myself, and sometimes they involve other people.

Am I holding onto opinions, attitudes, and beliefs that could be limiting my possibilities? Are they leading to assumptions and misunderstandings that end up costing me and those around me? Remember, the quality of my relationship (and therefore, my career, my marriage, etc.) is directly linked to the quality of my conversations.

What are some areas where my context has either helped or hindered my relationships—at home or in the workplace?”

Artificial Intelligence

Artificial Intelligence (AI) can be divided into disciplines such as, but not limited to, Machine Learning (ML), Natural Language Processing (NLP), and Deep Learning (DL). ML involves the creation of computers and software that are operable to learn from data, and then apply that knowledge to brand new data sets. DL creates neural networks, designed to resemble the human brain, and is used to process data including, but not limited to, sounds and images. AI cannot function without data. “Big Data” refers to the massive sets of data that are required and available for AI. Big Data sets are operable to be structured data including, but not limited to, transactional data in a relational database, and less structured or unstructured data, including, but not limited to, images, email data, and/or sensor data. These data sets are operable to be analyzed to find patterns, trends, and facilitate making future predictions.

ML algorithms provide effective automated tools for data collection, analysis, and integration. When combined with cloud computing power, ML enables fast and thorough processing and integration of large amounts of various information. ML algorithms are operable to be applied to every element of a Big Data operation including, but not limited to, data labeling and/or segmentation, data analytics, diagnostics, planning, prediction, and/or scenario simulation.

In one embodiment, AI, ML, and/or DL is used to provide intelligence and information about stress based on the data collected by the at least one data source.

In one embodiment, the system utilizes Artificial Intelligence (AI). In one embodiment, the system utilizes deploys Machine Learning (ML). In another embodiment, the system utilizes Deep Learning (DL). In yet another embodiment, the system utilizes AI, ML, DL, and/or combinations thereof.

AI, ML, DL, and/or Big Data enable the system to gather data intelligence, forecast, map, and/or provide market intelligence. In a preferred embodiment, AI, ML, DL, and/or Big Data algorithms are encompassed in the learning engine and/or the analytics engine of the remote server.

In one embodiment, the AI and/or the ML is operable to provide recommendations and present the recommendations through the GUI. In another embodiment, the AI and/or the ML is operable to automatically determine resources that best relate and/or be most effective to the situation presented. Data operable to be input to determine the most effective resources and conversation models includes, but is not limited to, data collected by the data source. In one embodiment, the AI and/or the ML is operable to use historical data to identify trends in the stressors, behaviors, etc., to make predictions as to future conditions to pre-emptively provide notifications and/or resources regarding upcoming events, conditions, and/or threats related to the stress levels of the user. In one embodiment, that AI and/or the ML includes one or more natural language processing (NLP) algorithm. Additional details about NLP and its abilities can be found in U.S. Patent Publication No. 2021/0350073, which is incorporated herein by reference in its entirety. The one or more NLP algorithm is operable to analyze audio data and/or text data to provide analysis of the meaning of speech and/or language captured in the audio data or written in text as a means to provide and present the most effective resources and conversation models; preferably, the resources presented are personalized or customized for at least one user. In a preferred embodiment, the at least one NLP algorithm is operable to receive feedback as a method of learning as a means to improve its performance. In one embodiment, the NLP algorithm is further operable to create a response based on voice and/or text input. In one embodiment, this response mimics the response a human would present in a human conversation. In one embodiment, this NLP algorithm is implemented in the form of a chatbot that is operable to be accessed through the GUI to answer questions regarding stressful situations. Input for the chatbot is operable to be audio and/or text input received through the GUI. In the present disclosure, data including audio data and/or text data that is operable to be interpreted by the chatbot is referred to generally as chat data. In some embodiments, the chatbot is further operable to process image and/or video inputs as a means to providing extra accessibility to individuals with disabilities. The chatbot is operable to output text and/or audio in form of speech to provide answers to the inputs. In one embodiment, the chatbot further provides and presents resources and/or conversation models associated with the questions/input.

In a preferred embodiment, the AI employed by the system includes a conversational AI. Conversational AI refers generally to technologies such as, but not limited to chatbots, virtual agents, and other non-human programs operable to receive and respond to input in the form of language. Input into a conversational AI is operable to include text and/or voice data. In a preferred embodiment, the conversational AI employed by the system is operable to identify and/or classify a scenario that a user is going through. In one embodiment, the conversational AI is operable to identify which scenario, from a predetermined list of scenarios, the input received by the AI most likely aligns. As a non-limiting example, the conversational AI is operable to analyze the input “My mom just yelled at me” and determine the input most closely aligns with scenario “Parent-Child Conflict.” From this, the conversational AI is then operable to recommend content and resources related to the scenario that is likely to be helpful in navigating the interaction, lowering the stress levels of the user.

Preferably, the AI is operable to implement different techniques into its analysis of the input. In one embodiment, the AI implements sentiment analysis to determine which scenario is most likely to align with the input. Sentiment analysis is operable to identify the sentiment of the input and determine the tone of the input such as, but not limited to, positive, negative, or neutral. Alternatively or additionally, the AI is operable to implement synonym mapping to improve its accuracy. Alternatively or additionally, the AI is operably to co-relate the response of the user with matching scenario questions based on the input by scoring the relevancy of each scenario question to the input. Preferably, the AI is operable to implement sentiment analysis, assigning relevancy score, and synonym mapping to provide the user account with the resources likely to be the most highly relevant to the input.

In one embodiment, the AI is operable to analyze population data to predict the stress type associated with a user account. As a non-limiting example, stress levels associated with a user account demonstrate a significant difference in personal “at home” events with higher stress levels associated with the user account when the data shows the user is likely at home. Through the AI and coaching, the platform is operable to determine at a high probability interval that the person is having issues setting priorities and managing expectations at work and at home. The platform can then prompt the user with proper questions around this predicted issue and known outcome based on user data and machine learning feedback loops.

Data Processing

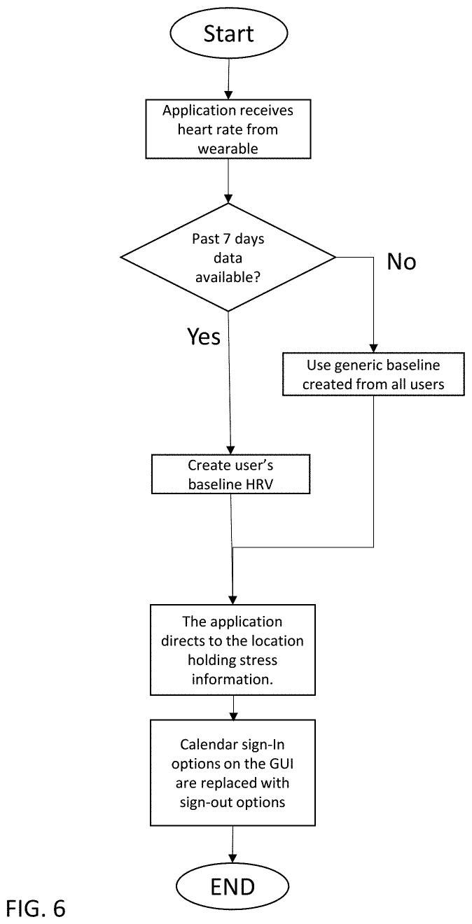

Data collected by the system is further operable to be processed to provide valuable insights and/or resources. In a preferred embodiment, the data is collected and processed to determine an associated stress level. Stress levels can be determined using any combination of data available to the system indicative of high stress levels such as, but not limited to, high heart rate, increased skin conductivity, increase in respiratory rate, etc. FIG. 6 illustrates a method for measuring stress levels according to one embodiment of the present invention. In a preferred embodiment, stress levels are measured using the method illustrated in FIG. 6. First, heart rate data is retrieved from at least one wearable and/or at least one application associated with at least one wearable. If the system has the past seven days of data available, the platform creates a baseline heart rate variability based on this data. If the past seven days of data is not available, a generic baseline heart rate variability is made from all users. Finally, the platform assigns a stress level based on a change in comparison to the baseline heart rate variability determined in the previous step. A high degree of change is associated with a high level of stress while a heart rate variability close or at the baseline is associated with low stress. Stress with respect to the time of day is monitored and stored by the platform to track stress throughout the day.

Data regarding stress levels determined by the platform is further operable to be cross-referenced with data retrieved from the calendar application. Advantageously, this provides context regarding the stress level detected by the platform. In a non-limiting example, the platform detects increased stress levels between the hours of 2:00 PM and 3:00 PM. Upon cross-referencing with data from the calendar application, the platform determines the user was at her mother-in-law's house. This information is displayed using the GUI and the platform further implements AI to determine and present resources helpful in navigating the situation.

In one embodiment, the data collected further includes location data. Advantageously, this allows for more data to be collected by the platform in reference to the context of a stressful situation. Additionally, location data is operable to be used as a means to determine the identity of individuals in close proximity to the user during a stress event. For example, each electronic device operable to provide location data is operable to be used as a unique identifier allowing for the determination of who else was around during the stress event using beacon and other geolocational technologies. This data is operable to be provided to the AI to increase the relevance and effectiveness of the resources presented. Altogether, data relating to the location and calendar is operable to be combined to tag stressful events with data regarding the possible cause of the stressful event.

In some embodiments, data collected by the platform is further processed with noise reduction and/or noise removal techniques. For example, the platform collects stress data associated with the user account that is particularly high due to a highly elevated heart rate. Based on the biometric data, the platform is operable to determine the highly elevated heart rate is caused by exercise being performed by the user. This data, although indicative of high stress, is not identified as a stress event by the platform since the stress experienced by the user is not due to a social-related stressor and is instead caused by a physical stressor. In one embodiment, noise removal is performed by setting certain criteria for noise stress events that should not be identified as stress event.

Criteria for categorizing a stress event include, but are not limited to, unreasonably high biometric data and calendar data indicative of likely non-social stressors. Alternatively or additionally, noise stress events are operable to be determined by integrating data from third-party sources. As a non-limiting example, the platform retrieves from a third-party source data indicating that a user associated with the elevated stress data indicated that the user was participating in exercise during that time. Therefore, the platform determines that the high stress data it retrieved was likely not caused by social stress but was likely caused by physical stress, and therefore is not identified as a stress event. Alternatively or additionally, the platform is operable to classify a stress event as a noise stress event by sending a prompt to the user account for confirmation by the user. In such embodiments, the platform determines a high stress event was likely a noise stress event by one or more of the methods disclosed herein. The platform is further operable to prompt the user to confirm the high stress event was a noise stress event. If the platform receives an indication that it was a noise stress event, it is not classified as a stress event. If the platform receives indication the high stress event was not a noise stress event, it is classified as a stress event and further processed by the platform.

Data Analytics

The present invention further provides data analytics and insightful information regarding the at least one user account. Data related to the at least one user account (e.g., data gathered through biometric data, calendar data, stress data, and/or location data) retrieved by the at least one electronic device. The data related to the at least one user account is further analyzed and operable to be presented, for example, via notifications, alerts, and/or reports. Examples include, but are not limited to, patterns involving historical stress data, historical calendar data, location data, etc.

Alternatively or additionally, data regarding stress associated with a user account is operable to be used to determine predictive analytics. In one embodiment, the platform is operable to analyze historical data and determine pattern in stress data associated with the user account. This data is then operable to provide insights regarding how stress data associated with a user account is operable to behave in the future. In one embodiment, the platform is operable to send at least one notification to the user account when the user is likely to experience a stressful event in the near future. In one embodiment, this prediction is determined by analyzing calendar data associated with the user account to determine future events associated with the user account. historical stress data and calendar data is further analyzed to determine if any events with similar names, attendees, etc. were associated with high levels of stress in the historical stress data. If so, a notification is sent to the user account at a predetermined time prior to the predicted stressful event. Alternatively or additionally, other data sources including, but not limited to, location data, time data, etc. are operable to be implemented in the determination of future upcoming stressful events.

In further embodiments, proactive recommendations are operable to be provided to the user account based on stressful locations and/or timeslots. As a non-limiting example, the platform collects data associated with the user account indicating the user is in a location the is often associated with high stress data. The platform is operable to use this data to further provide a recommendation to the user account. Alternatively or additionally, data relating to the current time is operable to be used to provide a recommendation. As a non-limiting example, the platform collects data regarding the stress level associated with a user account and determines a user is stressed at approximately the same time every day. In response, the platform provides a recommendation to the user such as, but not limited to, an exercise and/or a resource aimed to lower the stress associated with the user account before that timeslot to aid in managing the stress experienced at during the specific timeslot.

Furthermore, in embodiments wherein collected data includes GPS and/or location data including but not limited to beacons and other user interactions, analytics relating to such data are operable to be derived and presented through the GUI.

Application Flows

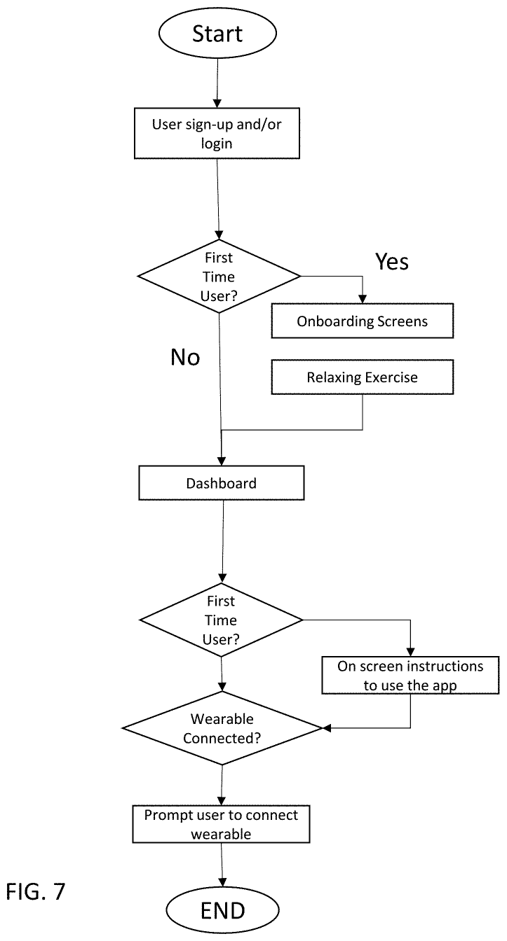

FIG. 7 illustrates a sign up and login method according to one embodiment of the present invention. First, the applications prompts for sign up and/or login by the through the GUI. If the user is a first-time user, the application proceeds through a series of onboarding screens to input relevant information for building a user profile such as name, age, etc. Next, the application present first time users with a relaxing exercise before directing them to the dashboard. If the user is not a first-time user, the application directs away from the login screen straight to the dashboard. From the dashboard, first-time users are presented with on screen instructions relating to how the application should be used. Next, checks are performed to see if at least one stress tracking wearable is connected linked to the application. If not, the application prompts the user to connect at least one wearable.

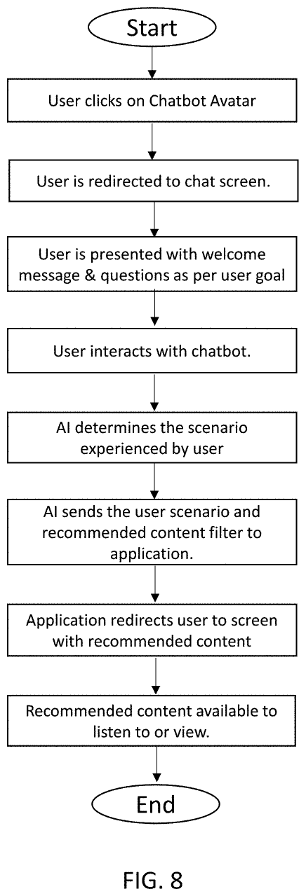

FIG. 8 illustrates a method for accessing content using a chatbot according to one embodiment of the present invention. First, the chatbot avatar is activated using the GUI. The platform then redirects the to the chat screen. The platform then presents a welcome message and questions regarding the goals of the user through the GUI. The chatbot is then operable to prompt for input. Any input is then sent to the AI for the determination of the scenario based on the received input. The AI is further operable to send scenarios and associated recommended content to the application. The application then redirects from the chatbot interface to the recommended content. The recommended content is then operable to be listened to and/or viewed through the GUI.

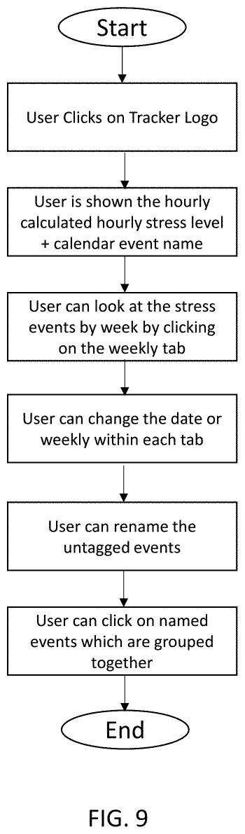

FIG. 9 illustrates a method for accessing a stress tracker according to one embodiment of the present invention. In a first step, the tracker logo is selected through a GUI. Then, the application presents the hourly calculated stress level along with the event name for the time obtained from the calendar data. The platform is operable to further present the stress events in different views including, but not limited to, a weekly view, an hourly view, etc. The application is operable to change views based on input received through the GUI. The application is further operable to receive input to rename untagged events and group stress events together based on at least one name.

Interfaces

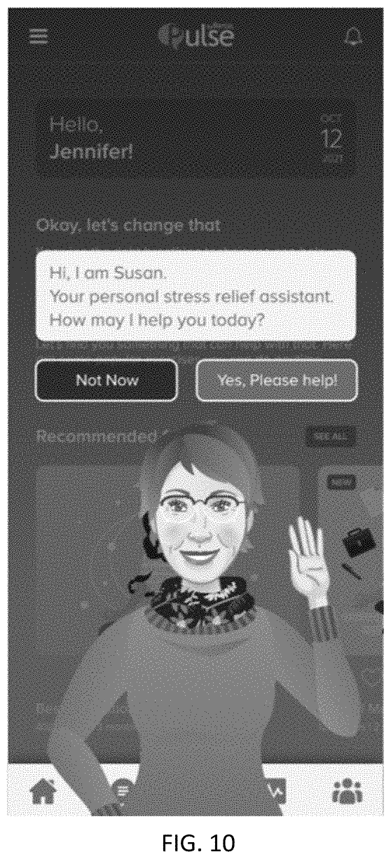

FIG. 10 illustrates an example of a personal assistant GUI for a stress management platform according to one embodiment of the present invention. The personal assistant GUI is operable to receive input as to the current state of a user. The platform prompts the user to input a current state into the personal assistant GUI. If the platform receives input of not now, the GUI returns to a dashboard screen. Alternatively, if the platform receives input that the user needs assistant, the platform will direct the user to a chatbot GUI.

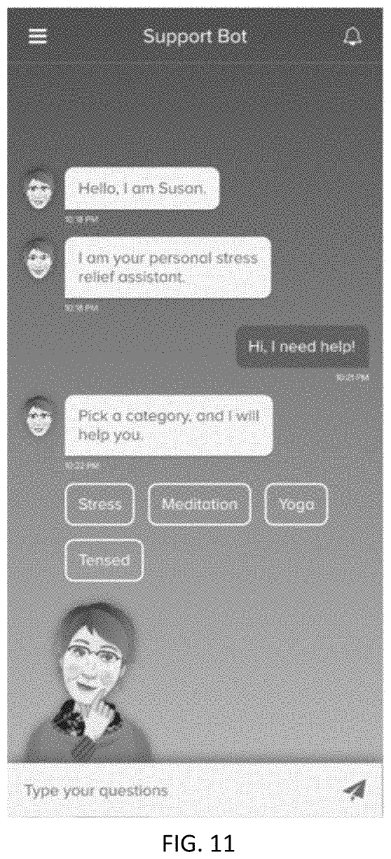

FIG. 11 illustrates an example of a chatbot GUI for a stress management platform according to one embodiment of the present invention. As described herein, the chatbot is operable to receive text and/or audio input and determine characteristics of the input such as, but not limited to, sentiment, meaning, etc. Upon interaction with the chatbot, the platform will direct the user account to resources most likely to be helpful with the information presented by the user.

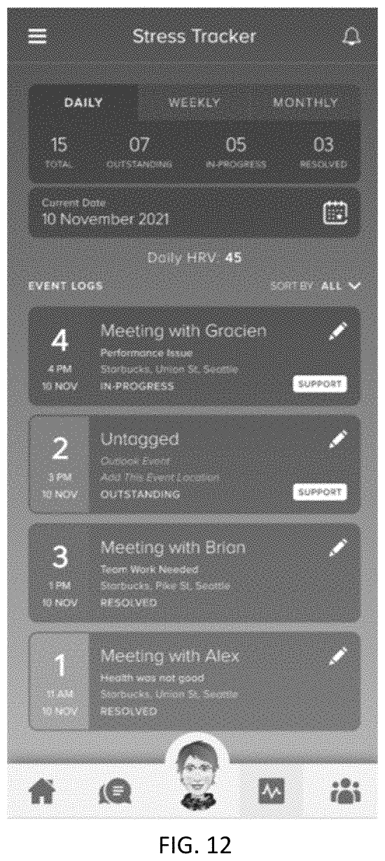

FIG. 12 illustrates an example of a dashboard GUI for a stress management platform according to one embodiment of the present invention. The dashboard GUI is operable to display data relating to past stressful events including time, determined stress level, tags based on calendar data and/or user input, location, and resolution status. Further, the dashboard is operable to be manipulated to display data in a daily formation, weekly format, or monthly format. In one embodiment, the dashboard GUI includes graphs of biometric data, such as line graphs of weekly HRV showing average, median, low, or high HRV by day and daily HRV showing average, median, low, or high HRV by hour. Any other biometric data or type of data recited herein is operable to be represented over any time period in any visual format, including graphs, charts, etc.

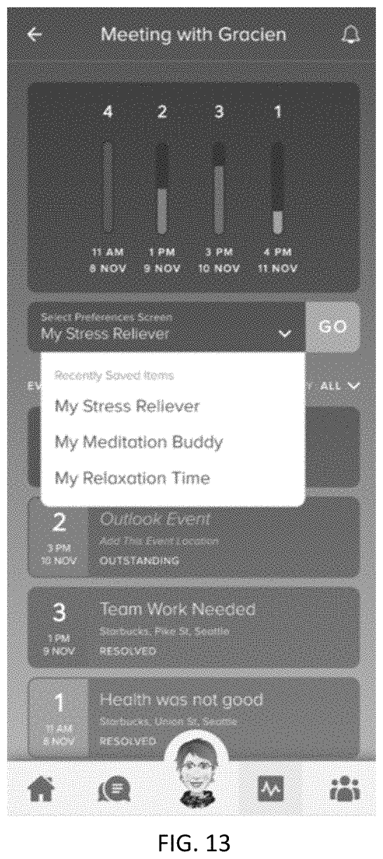

FIG. 13 illustrates an example of a stress event detail GUI for a stress management platform according to one embodiment of the present invention. The stress event detail GUI is operable to preset data relating to a stress event tagged with a certain tag. Further the stress event detail GUI is operable to provide a user with multiple options regarding immediate stress reduction techniques to aid in lowering the immediate stress of a user.

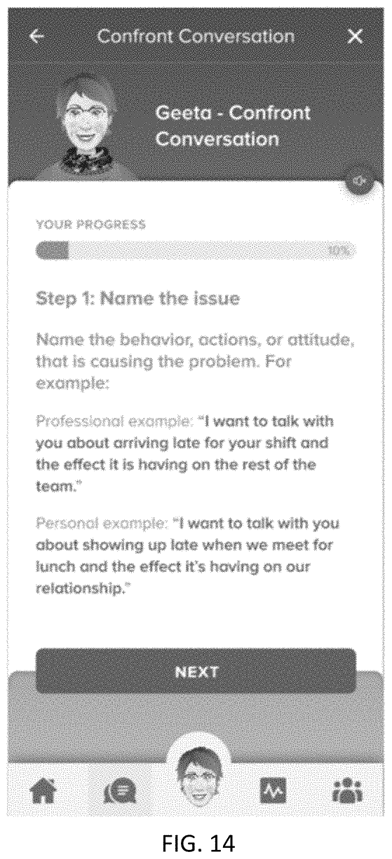

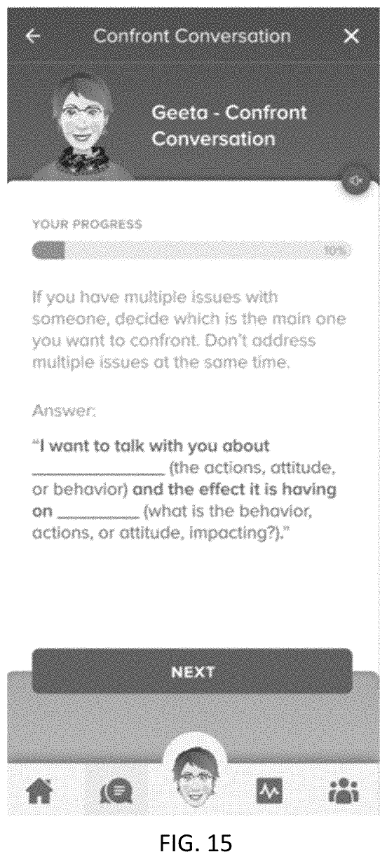

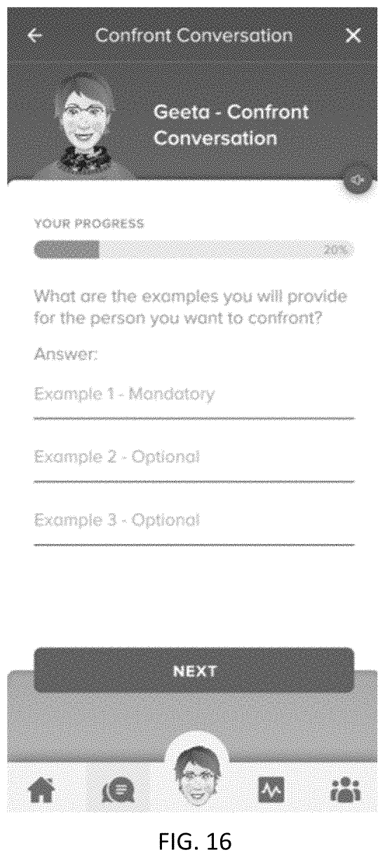

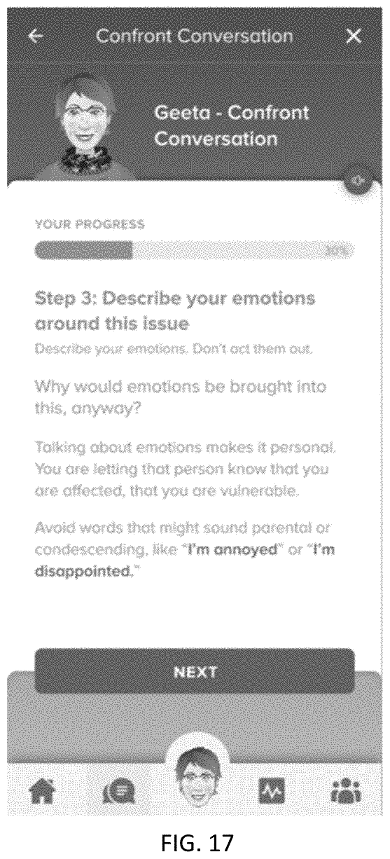

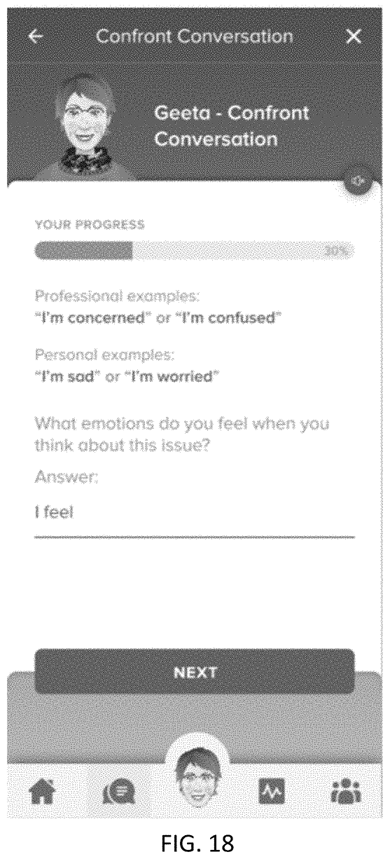

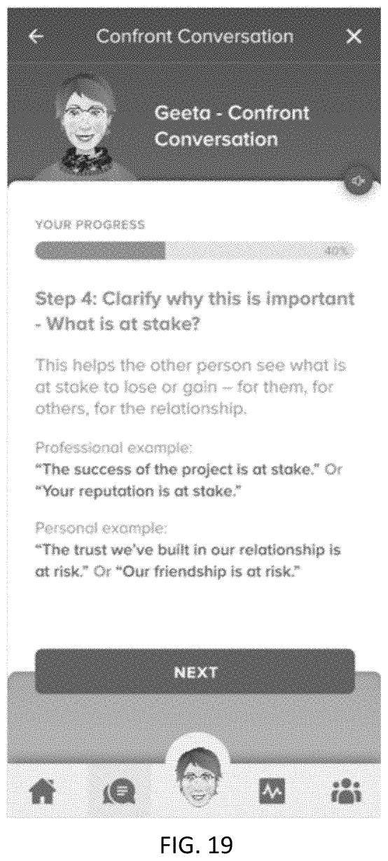

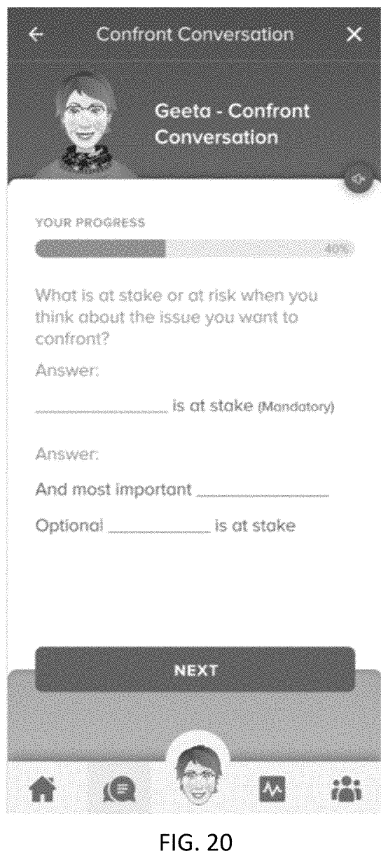

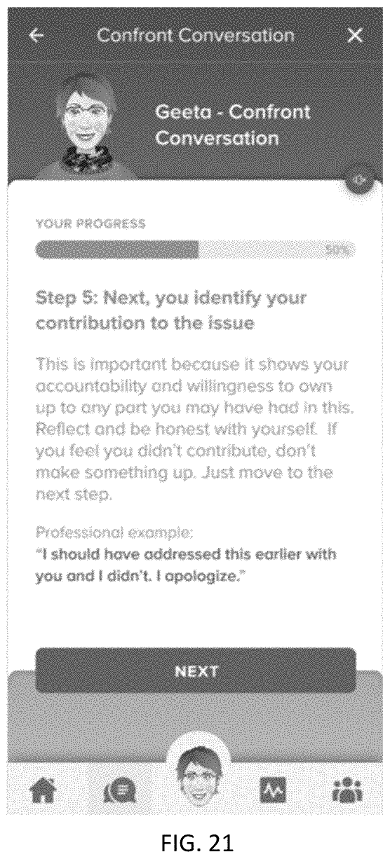

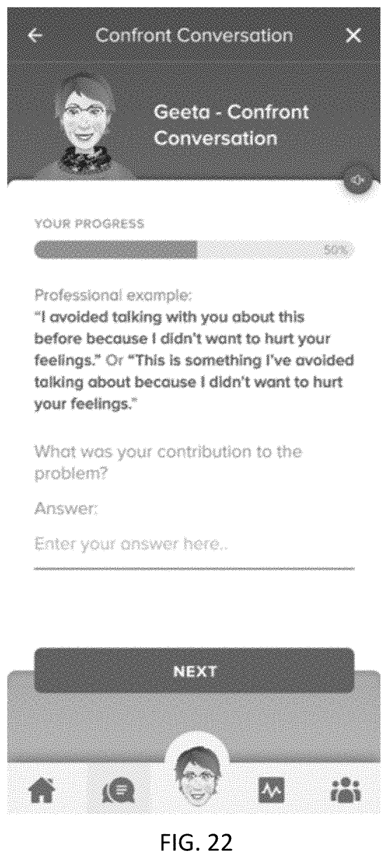

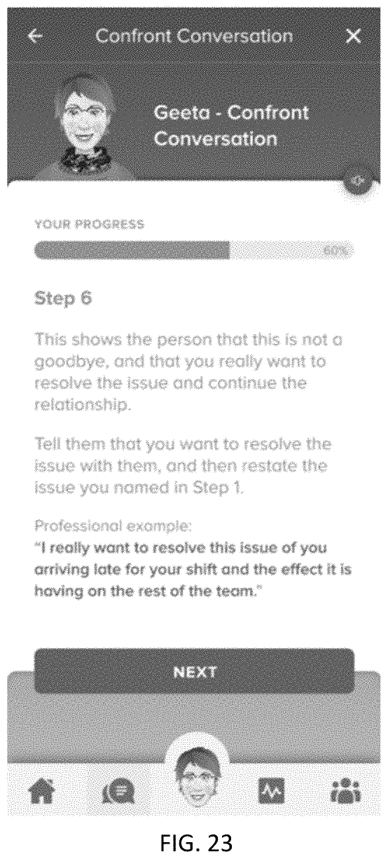

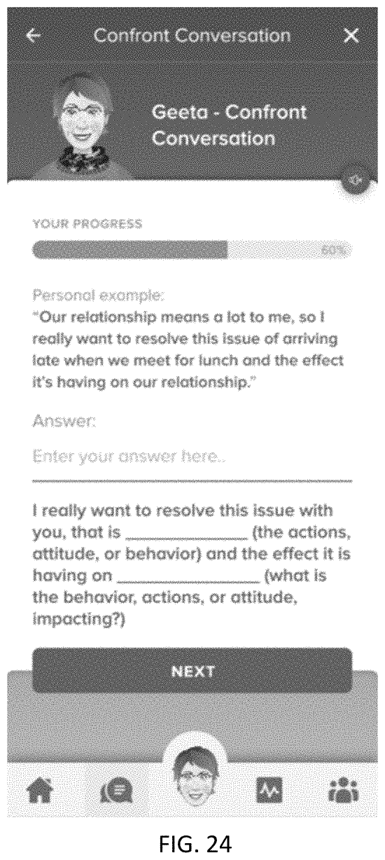

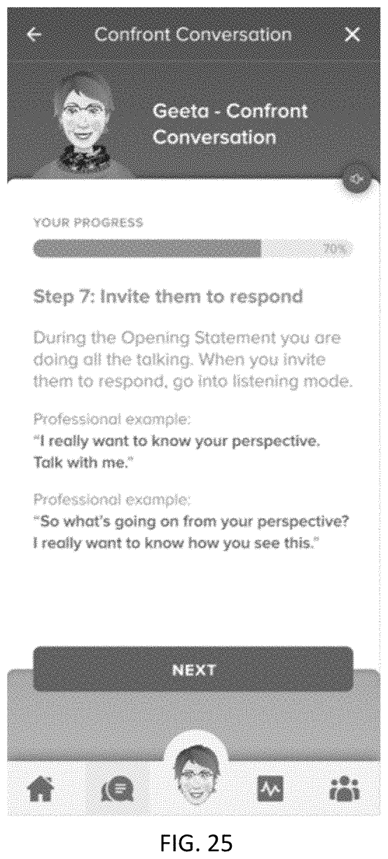

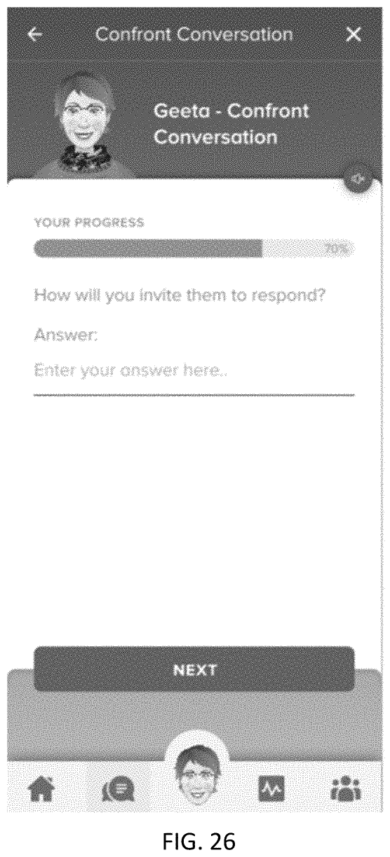

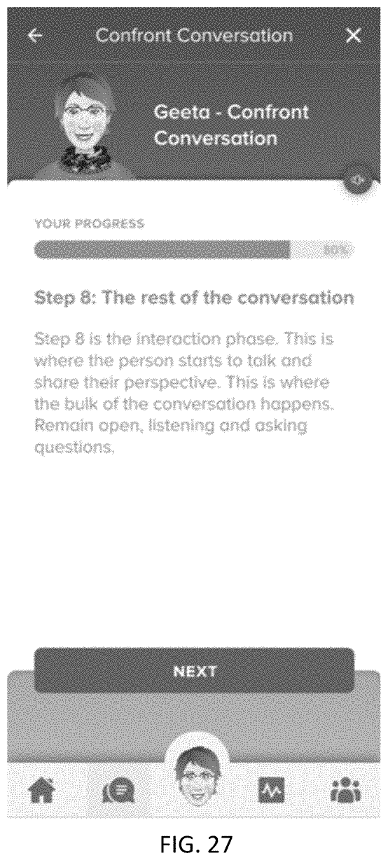

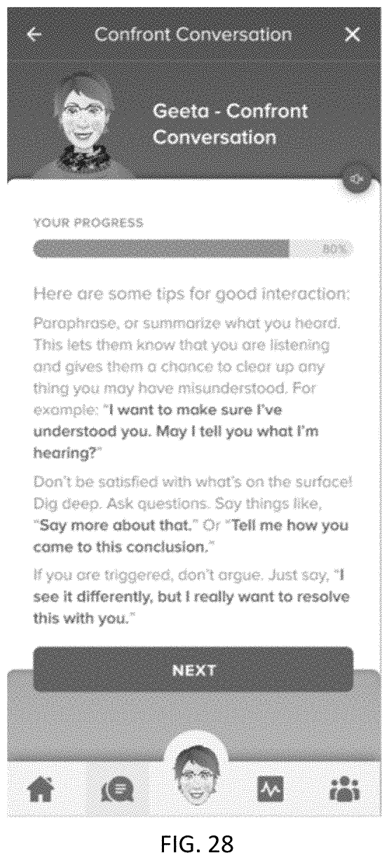

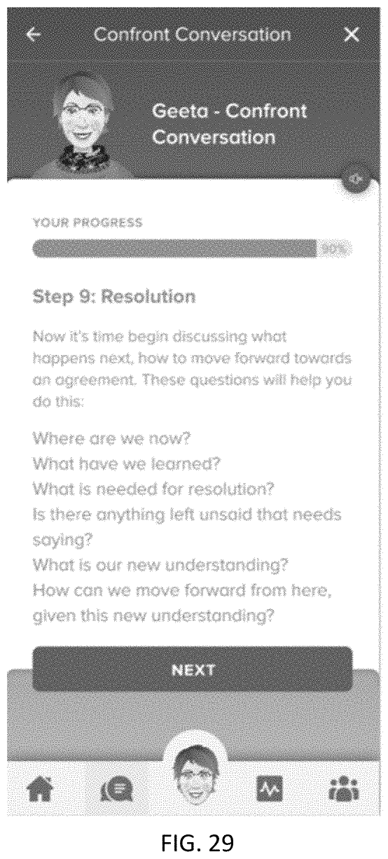

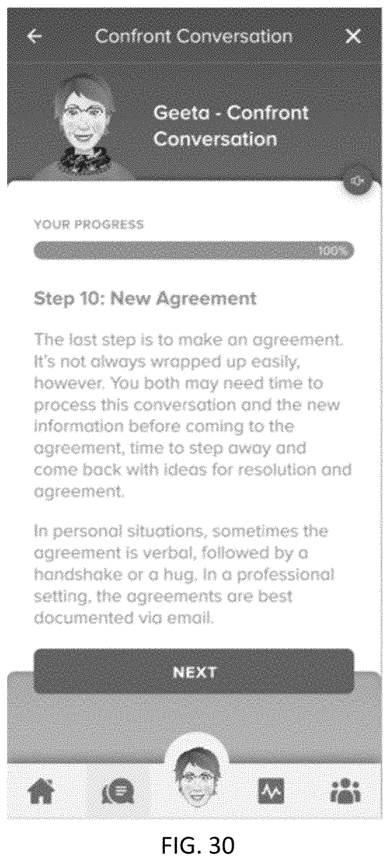

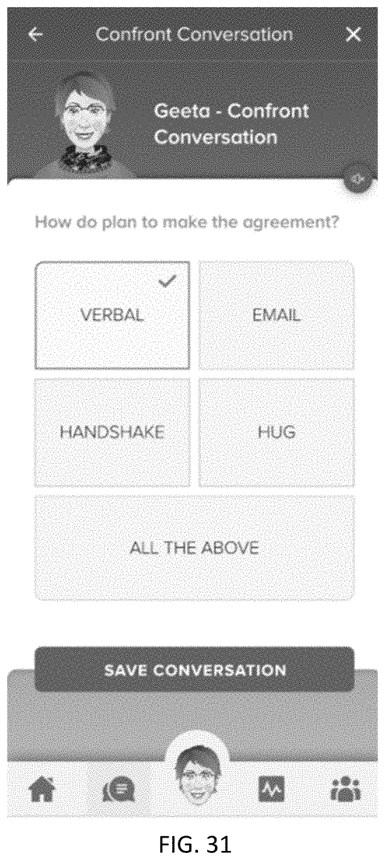

FIGS. 14-31 illustrate example confrontation conversation model GUIs for a stress management platform according to one embodiment of the present invention. Such GUIs are operable to be provided by the AI in the event the AI determines confrontation is the optimal way of handling a stress inducing conflict. The conversation model prompts the user for information regarding the confrontation (i.e. what the conflict is about, strategies to employ, etc.) in order to train the user to more effectively handle the confrontation. In one embodiment, based on the input received, the conversation model is operable to adjust the information provided based on the input. This allows for the conversation model to provide more useful training tailored more specifically to the situation of the user. Once the platform has received input for each step, the conversation model is operable to be saved as shown in FIG. 31 so it can be accessed by the user account at a later time in the event the user feels the need to review the material to be more prepared for the confrontation.

Although many examples of the present invention are generally directed to managing stress during conversations by providing recommendations, the present invention is also operable to be utilized in other scenarios and with other types of relationships than those explicitly described herein. Although the term “counseling” is utilized throughout the present application, one of ordinary skill in the art will understand that the present invention is operable to be utilized for therapy in addition to or in place of counseling. In one embodiment, the systems and methods of the present invention are operable to be used for cognitive-behavioral therapy, dialectical behavior therapy, interpersonal therapy, mentalization-based therapy, psychodynamic therapy, emotion-focused therapy, family therapy, and mindfulness-based therapy. The present invention is operable to implement conversation models and provide resources recited herein such as blogs, Q&A, simulations, podcasts, etc. in therapy embodiments. The present invention is also operable to use the same data sources used for counseling embodiments for therapy embodiments, including body sensors, calendar applications, and location sensors. A connection with a therapist is operable to be provided directly through the platform (i.e. through video chat, voice chat, etc.) and/or by providing contact information for a recommended therapist. The same engines including artificial intelligence engines, analytics engines, and alert engines are also operable to be used for therapy embodiments. One of ordinary skill in the art will understand that FIGS. 10-31 are operable to be adapted for and utilized in therapy embodiments.

In one embodiment, the systems and methods of the present invention are utilized in a coaching environment, such as for coaching in a sport, coaching for vocals or a musical instrument, coaching for acting or performance, coaching for public speaking, and/or coaching for games such as chess, poker, backgammon, etc. The present invention is operable to implement conversation models and provide resources recited herein such as blogs, Q&A, simulations, podcasts, etc. in coaching embodiments. The present invention is also operable to use the same data sources used for counseling embodiments for coaching embodiments, including body sensors, calendar applications, and location sensors. A connection with a coach is operable to be provided directly through the platform (i.e. through video chat, voice chat, etc.) and/or by providing contact information for a recommended coach. The same engines including artificial intelligence engines, analytics engines, and alert engines are also operable to be used for coaching embodiments. One of ordinary skill in the art will understand that FIGS. 10-31 are operable to be adapted for and utilized in coaching embodiments.

The systems and methods of the present invention are also operable to be utilized for a variety of personal or professional conversations or situations, such as for relationships between friends, relationships between spouses, relationships between family members, relationships between team mates on sports teams, etc.

Location data is created in the present invention using one or more hardware and/or software components. By way of example and not limitation, location data is created using the Global Positioning System (GPS), low energy BLUETOOTH based systems such as beacons, wireless networks such as WIFI, Radio Frequency (RF) including RF Identification (RFID), Near Field Communication (NFC), magnetic positioning, and/or cellular triangulation. By way of example, location data is determined via an Internet Protocol (IP) address of a device connected to a wireless network. A wireless router is also operable to determine identities of devices connected to the wireless network through the router, and thus is operable to determine the locations of these devices through their presence in the connection range of the wireless router.

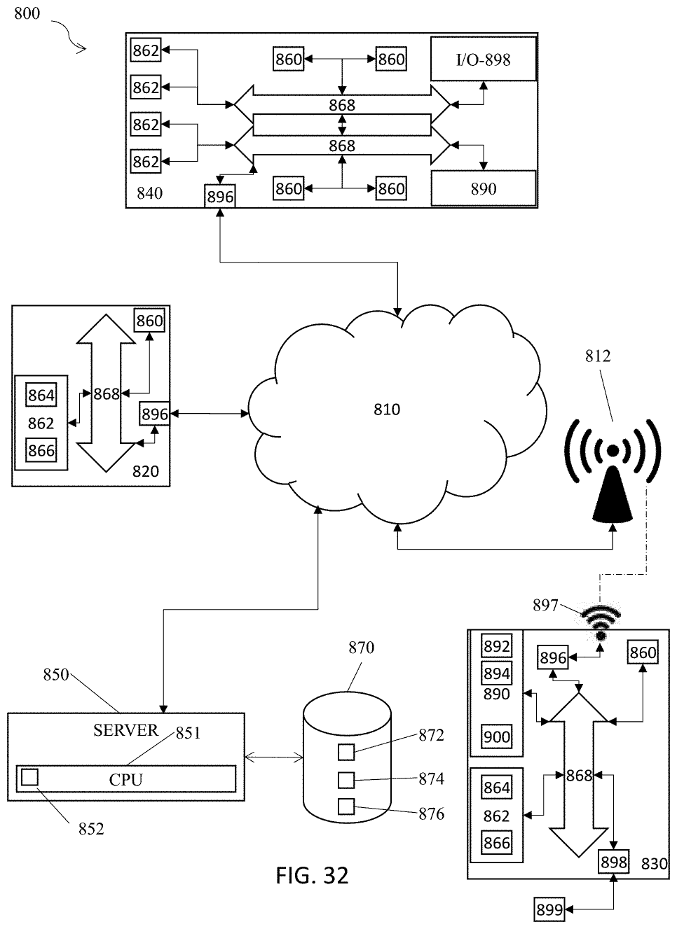

FIG. 32 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 is operable to house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 is operable to be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 is operable to additionally include components such as a storage device 890 for storing the operating system 892 and one or more application programs 894, a network interface unit 896, and/or an input/output controller 898. Each of the components is operable to be coupled to each other through at least one bus 868. The input/output controller 898 is operable to receive and process input from, or provide output to, a number of other devices 899, including, but not limited to, alphanumeric input devices, mice, electronic styluses, display units, touch screens, signal generation devices (e.g., speakers), or printers.

By way of example, and not limitation, the processor 860 is operable to be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 32, multiple processors 860 and/or multiple buses 868 are operable to be used, as appropriate, along with multiple memories 862 of multiple types (e.g., a combination of a DSP and a microprocessor, a plurality of microprocessors, one or more microprocessors in conjunction with a DSP core).

Also, multiple computing devices are operable to be connected, with each device providing portions of the necessary operations (e.g., a server bank, a group of blade servers, or a multi-processor system). Alternatively, some steps or methods are operable to be performed by circuitry that is specific to a given function.

According to various embodiments, the computer system 800 is operable to operate in a networked environment using logical connections to local and/or remote computing devices 820, 830, 840 through a network 810. A computing device 830 is operable to connect to a network 810 through a network interface unit 896 connected to a bus 868. Computing devices are operable to communicate communication media through wired networks, direct-wired connections or wirelessly, such as acoustic, RF, or infrared, through an antenna 897 in communication with the network antenna 812 and the network interface unit 896, which are operable to include digital signal processing circuitry when necessary. The network interface unit 896 is operable to provide for communications under various modes or protocols.

In one or more exemplary aspects, the instructions are operable to be implemented in hardware, software, firmware, or any combinations thereof. A computer readable medium is operable to provide volatile or non-volatile storage for one or more sets of instructions, such as operating systems, data structures, program modules, applications, or other data embodying any one or more of the methodologies or functions described herein. The computer readable medium is operable to include the memory 862, the processor 860, and/or the storage media 890 and is operable be a single medium or multiple media (e.g., a centralized or distributed computer system) that store the one or more sets of instructions 900. Non-transitory computer readable media includes all computer readable media, with the sole exception being a transitory, propagating signal per se. The instructions 900 are further operable to be transmitted or received over the network 810 via the network interface unit 896 as communication media, which is operable to include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system 800.

In one embodiment, the computer system 800 is within a cloud-based network. In one embodiment, the server 850 is a designated physical server for distributed computing devices 820, 830, and 840. In one embodiment, the server 850 is a cloud-based server platform. In one embodiment, the cloud-based server platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 is operable to not include all of the components shown in FIG. 32, is operable to include other components that are not explicitly shown in FIG. 32, or is operable to utilize an architecture completely different than that shown in FIG. 32. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

The invention claimed is:

1. A system for stress management comprising:
an application on at least one electronic device including a memory and a processor configured for network communication with at least one server computer comprising at least one database;
wherein the application is further operable to receive biometric data collected by at least one body sensor;
wherein the application is operable to analyze the biometric data and determine a stress level associated with the biometric data;
wherein the application is operable to receive calendar data from at least one calendar data source;
wherein the at least one server computer is operable to receive the biometric data and the calendar data from the application;
wherein the at least one server computer is operable to determine and retrieve at least one recommended resource from the at least one database based on the stress level and/or the calendar data;
wherein the at least one server computer is operable to send the at least one recommended resource to the application; and
wherein the application is operable to present the at least one recommended resource via a graphical user interface (GUI).

2. The system of claim 1, wherein the at least one body sensor comprises a respiration sensor, a heart rate sensor, a movement sensor, a brain wave sensor, a body temperature sensor, an analyte sensor, a blood pressure sensor, and/or an electrodermal activity sensor.

3. The system of claim 1, wherein the at least one body sensor is included in at least one wearable device.

4. The system of claim 1, wherein the at least one server computer determines the at least one recommended resource using an artificial intelligence engine and/or a machine learning engine.

5. The system of claim 4, wherein the at least one server computer is operable to train the artificial intelligence engine and/or machine engine by receiving feedback input through the GUI.

6. The system of claim 1, wherein the application is further operable to receive location data and/or chat data and send the location data and/or the chat data to the at least one server computer, wherein the at least one server computer is operable to determine and retrieve the at least one recommended resource from the at least one database based on the location data and/or the chat data.

7. The system of claim 1, wherein the application further comprises a chatbot operable to receive inputs providing details about a stressful situation, wherein the chatbot is further operable to provide resources in the form of conversation based on the details about the stressful situation.

8. The system of claim 1, wherein the at least one recommended resource includes a conversation model, counselor information, and/or a mindfulness exercise.

9. The system of claim 1, wherein the at least one calendar data source includes at least one external calendar platform.

10. The system of claim 1, wherein the stress level is further operable to be displayed via the GUI.

11. A system for stress management comprising:
an application on at least one electronic device including a memory and a processor configured for network communication with at least one server computer comprising at least one database;
wherein the application is further operable to receive biometric data collected by at least one body sensor;
wherein the application is operable to analyze the biometric data and determine a stress level associated with the biometric data;
wherein the application is operable to receive chat data from the at least one electronic device;
wherein the at least one server computer is operable to receive the biometric data and the chat data from the application;
wherein the at least one server computer is operable to determine and retrieve at least one recommended resource from the at least one database using an artificial intelligence engine and/or a machine learning engine based on the stress level and/or the chat data;
wherein the at least one server computer is operable to send the at least one recommended resource to the application; and
wherein the at least one server computer is operable to send the at least one recommended resource to the application.

12. The system of claim 11, wherein the application is operable to present the at least one recommended resource via a graphical user interface (GUI) on the at least one electronic device.

13. The system of claim 11, wherein the application is further operable to receive location data and/or calendar data and send the location data and/or the calendar data to the at least one server computer, wherein the at least one server computer is operable to determine and retrieve the at least one recommended resource from the at least one database based on the location data and/or the calendar data.

14. The system of claim 11, wherein the at least one server computer is operable to receive feedback from the application to train the artificial intelligence engine and/or the machine learning engine.

15. The system of claim 11, wherein the at least one recommended resource includes a conversation model, counselor information, a blog recommendation, a podcast, a video, diversity, equity, and inclusion (DE&I) content, anti-discrimination content, a simulation, and/or a mindfulness exercise.

16. The system of claim 11, wherein the at least one recommended resource is operable to be received by the electronic device and communicated as audio output.

17. A method for stress management comprising:
an application on an electronic device including a processor and a memory receiving data from at least one data source, wherein the data includes biometric data and calendar data;
the application analyzing the biometric data to determine a stress level associated with the data;
a server computer in network communication with the application on the electronic device determining and retrieving at least one recommended resource based on the data;
the server computer sending the at least one recommended resource to the application; and
the application presenting the at least one recommended resource via a GUI; and
wherein the biometric data is collected by at least one body sensor.

18. The method of claim 17, wherein the at least one recommended resource comprises a conversation model, counselor information, a blog recommendation, a podcast, a video, diversity, equity, and inclusion (DE&I) content, anti-discrimination content, a simulation, and/or a mindfulness exercise.

19. The method of claim 17, wherein the data further comprises chat data and/or location data.

20. The method of claim 17, wherein determining the at least one recommended resource is achieved by implementing artificial intelligence and/or machine learning.
